(19) United States
(12) Patent Application Publication
(10) Pub. No.: US 2009/0221582 A1
(43) Pub. Date: Sep. 3, 2009

(54) Piperazine Derivatives And Their Use In Therapy

(75) Inventors: Nathaniel Julius Thomas Monck, Berkshire (GB); James Edward Paul Davidson, Berkshire (GB); Claire Louise Nunns, Berkshire (GB); Stephen David Reeves, Berkshire (GB)

Correspondence Address: BANNER & WITCOFF, LTD., 1100 13th STREET, N.W., SUITE 1200, WASHINGTON, DC, 20005-4051, US

(73) Assignee: Vernalis (R&D), Berkshire (GB)

(21) Appl. No.: 12/094,181

(22) PCT Filed: Nov. 17, 2006
(86) PCT No.: PCT/GB2006/004300

§ 371 Date: Dec. 23, 2008

(30) Foreign Application Priority Data
Nov. 19, 2005 (GB) 0523609.6

(51) Int. Cl. A61K 31/5377; A61K 31/497; A61K 31/4965; C07D 413/02; C07D 401/02; C07D 241/04

(52) U.S. Cl. 514/235.8; 514/253.01; 514/255.01; 544/121; 544/360; 544/386

(57) ABSTRACT

Compounds of formula (I) and their use in therapy, particularly for the treatment of a disorder mediated by CB₁ receptors wherein R₁ is a radical of formula -(Alk¹)ₘ-(NH)ₚ-(Alk²)ₙ-Q wherein m, n and p are independently 0 or 1, Alk¹ and Alk² are straight or branched chain divalent C₁-C₆ alkylene or C₂-C₆ alkenylene radicals, and Q is (i) hydrogen, except in the case where m, n and p are each 0, or (ii) an optionally substituted carbocyclic or heterocyclic group; R₂ is hydrogen, C₁-C₃ alkyl, cyclopropyl, or —CF₃; Ring A is a phenyl or 5- or 6-membered heteroaryl ring either of which is optionally substituted; Ar is a phenyl or 5- or 6-membered heteroaryl ring either of which is optionally substituted; L is —CH₂—, —C(═O)—, —NH—, —O—, —S—, —SO—, —SO₂—, —(CH₂)₂—, —CH═CH—, —OCH₂—, —CH(CH₃)—, or —NH—CH₂—; s is 1 and W is an optionally substituted N-containing heterocyclic ring of 5 to 7 ring atoms; or s is 0 and W is an optionally substituted N-containing saturated heterocyclic ring of 5 to 7 ring atoms, or an N-containing heteroaryl ring of 5 or 6 ring atoms substituted by at least one substituent selected from amino, C₁-C₆ alkylamino or cyano; or a pharmaceutically acceptable salt, hydrate or solvate thereof.

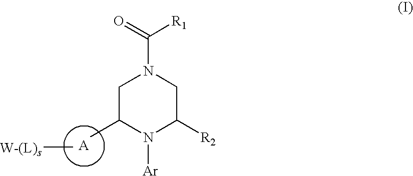

This invention relates to substituted piperazine compounds having CB1 antagonistic activity, to the use of such compounds in medicine, in relation to the treatment of disorders which are responsive to antagonism of the cannabinoid CB1 receptor such as obesity and other eating disorders associated with excessive food intake, and to pharmaceutical compositions containing such compounds.

### BACKGROUND TO THE INVENTION

It has been recognised that obesity is a disease process influenced by environmental factors in which the traditional weight loss methods of dieting and exercise need to be supplemented by therapeutic products (S. Parker, “Obesity: Trends and Treatments”, Scrip Reports, PJB Publications Ltd, 1996).

Whether someone is classified as overweight or obese is generally determined on the basis of their body mass index (BMI) which is calculated by dividing body weight (kg) by height squared (m²). Thus, the units of BMI are kg/m² and it is possible to calculate the BMI range associated with minimum mortality in each decade of life. Overweight is defined as a BMI in the range 25-30 kg/m², and obesity as a BMI greater than 30 kg/m². There are problems with this definition in that it does not take into account the proportion of body mass that is muscle in relation to fat (adipose tissue). To account for this, obesity can also be defined on the basis of body fat content: greater than 25% and 30% in males and females, respectively.

As the BMI increases there is an increased risk of death from a variety of causes that is independent of other risk factors. The most common diseases with obesity are cardiovascular disease (particularly hypertension), diabetes (obesity aggravates the development of diabetes), gall bladder disease (particularly cancer) and diseases of reproduction. Research has shown that even a modest reduction in body weight can correspond to a significant reduction in the risk of developing coronary heart disease or diabetes.

Current compounds marketed as anti-obesity agents include Orlistat (Reductil®) and Sibutramine. Orlistat (a lipase inhibitor) inhibits fat absorption directly and tends to produce a high incidence of unpleasant (though relatively harmless) side-effects such as diarrhoea. Sibutramine (a mixed 5-HT/noradrenaline reuptake inhibitor) can increase blood pressure and heart rate in some patients. The serotonin releaser/reuptake inhibitors fenfluramine (Pondimin®) and dexfenfluramine (Redux™) have been reported to decrease food intake and body weight over a prolonged period (greater than 6 months). However, both products were withdrawn after reports of preliminary evidence of heart valve abnormalities associated with their use. There is therefore a need for the development of a safer anti-obesity agent.

There now exists extensive pre-clinical and clinical data supporting the use of CB1 receptor antagonists/inverse agonists for the treatment of obesity.

Preparations of marijuana (Cannabis sativa) have been used for over 5000 years for both medicinal and recreational purposes. The major psychoactive ingredient of marijuana has been identified as delta-9-tetrahydrocannabinol (delta-9-THC), one of a member of over 60 related cannabinoid compounds isolated from this plant. It has been demonstrated that delta-9-THC exerts its effects via agonist interaction with cannabinoid (CB) receptors. So far, two cannabinoid receptor subtypes have been characterised (CB1 and CB2). The CB1 receptor subtype is found predominantly in the central nervous system, and to a lesser extent in the peripheral nervous system and various peripheral organs. The CB2 receptor subtype is found predominantly in lymphoid tissues and cells. To date, three endogenous agonists (endocannabinoids) have been identified which interact with both CB1 and CB2 receptors (anandamide, 2-arachidonyl glycerol and noladin ether).

Genetically obese rats and mice exhibit markedly elevated endocannabinoid levels in brain regions associated with ingestive behaviour (Di Marzo et al. 2001 Nature 410: 822-825). Furthermore, increased levels of endocannabinoids are observed upon the fasting of normal, lean animals (Kirkham et al., British Journal of Pharmacology 2002, 136(4) 550-557). Exogenous application of endocannabinoids leads to the same physiological effects observed with delta-9-THC treatment, including appetite stimulation (Jamshida et al., British Journal of Pharmacology 2001, 134: 1151-1154), analgesia, hypolocomotion, hypothermia, and catalepsy.

CB1 (CB1−/−) and CB2 (CB2−/−) receptor knockout mice have been used to elucidate the specific role of the two cannabinoid receptor subtypes. Furthermore, for ligands such as delta-9-THC which act as agonists at both receptors, these mice have allowed identification of which receptor subtype is mediating specific physiological effects. CB1−/−, but not CB2−/−, mice are resistant to the behavioural effects of agonists such as delta-9-THC. CB1−/− animals have also been shown to be resistant to both the body weight gain associated with chronic high fat diet exposure, and the appetite-stimulating effects of acute food deprivation.

These findings suggest a clear role for both endogenous and exogenous cannabinoid receptor agonists in increasing food intake and body weight via selective activation of the CB1 receptor subtype.

The therapeutic potential for cannabinoid receptor ligands has been extensively reviewed (Exp. Opin. Ther. Pat. 1998, 8, 301-313; Exp. Opin. Ther. Pat. 2000, 10, 1529-1538; Trends in Pharm. Sci. 2000, 21, 218-224; Exp. Opin. Ther. Pat. 2002, 12(10), 1475-1489).

At least one compound (SR-141716A; Rimonabant) characterised as a CB1 receptor antagonist/inverse agonist is known to be in clinical trials for the treatment of obesity.

Clinical trials with the CB1 receptor antagonist rimonabant have also observed an antidiabetic action that exceeds that accounted for by weight loss alone (Scheen A. J., et al., Lancet, 2006 in press). CB1 receptor mRNA is located on α- and β-cells in the Islets of Langerhans and it has been reported that CB1 receptor agonists reduce insulin release from pancreatic beta cells in vitro in response to a glucose load (Juan-Pico et al, Cell Calcium, 39, (2006), 155-162). Consistent with this, Bermudez-Siva et al., (Eur J Pharmacol., 531 (2006), 282-284) have reported that CB1 receptor agonists increase glucose intolerance following ip injection of a glucose load to rats. This effect was reversed by a CB1 receptor antagonist that increased glucose tolerance in the test when given alone. Thus, the action of rimonabant may be due to a direct action on the pancreas. It is also possible that CB1 receptor antagonists affect insulin sensitivity indirectly via an action on adiponectin (Chandran et al., Diabetes care, 26, (2003), 2442-2450) which is elevated by CB1 receptor antagonists (Cota et al., J Clin Invest., 112 (2003), 423-431; Bensaid et al., Mol Pharmacol., 63 (2003, 908-914). Indeed, it has been reported that endocannabinoid levels are enhanced in the pancreas and adipose tissue of obese and diabetic mice and in the plasma and adipose tissue of obese or type 2 diabetic patients (Matias et al., J Clin Endocrinol and Metab., 91 (2006), 3171-3180) suggesting a possible causal role of elevated cannabinoid tone in the onset of type 2 diabetes.

There remains a medical need for low molecular weight CB1 receptor antagonists/inverse agonists with pharmacokinetic and pharmacodynamic properties making them suitable for use as pharmaceutical agents. There also remains a medical need for new treatments of disorders mediated by the CB1 receptor, particularly diabetes and eating disorders, and particularly obesity. The object of the present invention is to provide such pharmaceutical agents and treatments.

It has now been found that certain substituted piperazines show efficacy as anti-obesity and potentially anti-diabetic agents. These substituted piperazines have been shown to selectively bind to the CB1 receptor subtype with high affinity. Such compounds have been shown to dose-dependently block the effects of an exogenously applied cannabinoid receptor agonist (e.g. delta-9-THC) in mice. Furthermore, such compounds have been shown to reduce food intake and body weight gain in both rat and mouse models of feeding behaviour.

### BRIEF DESCRIPTION OF THE INVENTION

The present invention relates to a class of substituted piperazine compounds useful as CB1 antagonists, for example, for the treatment of obesity and/or diabetes. A core piperazine ring with aromatic or heteroaromatic substitution on one ring nitrogen, in addition to aromatic or heteroaromatic substitution on an adjacent ring carbon atom, are principle characterising features of the compounds with which the invention is concerned.

### DETAILED DESCRIPTION OF THE INVENTION

According to the present invention, there is provided a compound of formula (I) or a pharmaceutically acceptable salt, hydrate, solvate, or prodrug thereof:

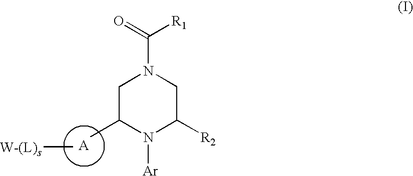

wherein

R₁ is a radical of formula -(Alk¹)ₘ-(NH)ₚ-(Alk²)ₙ-Q wherein
m, n and p are independently 0 or 1,
Alk¹ and Alk² are straight or branched chain divalent C₁-C₆ alkylene or C₂-C₆ alkenylene radicals, and
Q is (i) hydrogen, except in the case where m, n and p are each 0, or (ii) an optionally substituted carbocyclic or heterocyclic group;

R₂ is hydrogen, C₁-C₃ alkyl, cyclopropyl, or —CF₃;

Ring A is a phenyl or 5- or 6-membered heteroaryl ring either of which is optionally substituted;

Ar is a phenyl or 5- or 6-membered heteroaryl ring either of which is optionally substituted;

L is —CH₂—, —C(═O)—, —NH—, —O—, —S—, —SO—, —SO₂—, —(CH₂)₂—, —CH═CH—, —OCH₂—, —CH(CH₃)—, or —NH—CH₂—;

s is 1 and W is an optionally substituted N-containing heterocyclic ring of 5 to 7 ring atoms; or s is 0 and W is an optionally substituted N-containing saturated heterocyclic ring of 5 to 7 ring atoms, or an N-containing heteroaryl ring of 5 or 6 ring atoms substituted by at least one substituent selected from amino, C₁-C₆ alkylamino or cyano.

A particular subset of the invention is a compound of formula (I) or a pharmaceutically acceptable salt, hydrate, solvate, or prodrug thereof wherein

R₁ is a radical of formula -(Alk¹)ₘ-(NH)ₚ-(Alk²)ₙ-Q wherein
m, n and p are independently 0 or 1,
Alk¹ and Alk² are straight or branched chain divalent C₁-C₆ alkylene or C₂-C₆ alkenylene radicals, and
Q is (i) hydrogen, except in the case where m, n and p are each 0, or (ii) an optionally substituted carbocyclic or heterocyclic group;

R₂ is hydrogen, C₁-C₃ alkyl, cyclopropyl, or —CF₃;

Ring A is a phenyl or 5- or 6-membered heteroaryl ring either of which is optionally substituted;

Ar is a phenyl or 5- or 6-membered heteroaryl ring either of which is optionally substituted;

L is —CH₂—, —C(═O)—, —NH—, —O—, —S—, —SO—, —SO₂—, —(CH₂)₂—, —CH═CH—, or —OCH₂—;

s is 1 and W is an optionally substituted N-containing heterocyclic ring of 5 or 6 ring atoms; or s is 0 and W is an optionally substituted N-containing saturated heterocyclic ring of 5 or 6 ring atoms, or an N-containing heteroaryl ring of 5 or 6 ring atoms substituted by at least one substituent selected from amino, C₁-C₆ alkylamino or cyano.

The active compounds of formula (I) are antagonists at the cannabinoid-1 (CB₁) receptor and are useful for the treatment, prevention and suppression of diseases mediated by the CB₁ receptor. The invention is also concerned with the use of these compounds to selectively antagonise the CB₁ receptor and in the treatment of obesity, diabetes and other disorders.

As used herein, the term “antagonist” refers to a compound that is an antagonist and/or inverse agonist of the CB₁ receptor.

An antagonist is a compound which has no intrinsic modulatory activity, but produces effects by interfering with an endogenous agonist or inhibiting the actions of an agonist. An inverse agonist is a compound which acts on a receptor to reverse normal constitutive receptor activity. In the absence of constitutive activity, inverse agonists may behave as competitive antagonists. In functional assays, an inverse agonist is an agent which binds to the receptor but exerts an opposing pharmacological effect.

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers refers to a saturated hydrocarbon chain having from a to b carbon atoms and two unsatisfied valences.

As used herein the term “(Cₐ-C_{b})alkenyl” wherein a and b are integers refers to a straight or branched chain alkenyl moiety having from a to b carbon atoms having at least one double bond of either E or Z stereochemistry where applicable. The term includes, for example, vinyl, allyl, 1- and 2-butenyl and 2-methyl-2-propenyl.

As used herein the term “divalent (Cₐ-C_{b})alkenylene radical” refers to a hydrocarbon chain having from a to b carbon atoms, at least one double bond, and two unsatisfied valences.

As used herein the term “cycloalkyl” refers to a saturated carbocyclic radical having from 3-8 carbon atoms and includes, for example, cyclopropyl, cyclobutyl, cyclopentyl, cyclohexyl, cycloheptyl and cyclooctyl.

As used herein the term “cycloalkenyl” refers to a carbocyclic radical having from 3-8 carbon atoms containing at least one double bond, and includes, for example, cyclopentenyl, cyclohexenyl, cycloheptenyl and cyclooctenyl.

As used herein the term “aryl” refers to a mono-, bi- or tri-cyclic carbocyclic aromatic radical. Illustrative of such radicals are phenyl, biphenyl and napthyl.

As used herein the term “carbocyclic” refers to a mono- or bi-cyclic radical whose ring atoms are all carbon, and includes monocyclic aryl, cycloalkyl, and cycloalkenyl radicals, provided that no single ring present has more than 8 ring members. A “carbocyclic” group includes a mono-bridged or multiply-bridged cyclic alkyl group.

As used herein the term “heteroaryl” refers to a mono-, bi- or tri-cyclic aromatic radical containing one or more heteroatoms selected from S, N and O. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in particular refers to a mono-, bi- or tri-cyclic non-aromatic radical containing one or more heteroatoms selected from S, N and O, to groups consisting of a monocyclic non-aromatic radical containing one or more such heteroatoms which is covalently linked to another such radical or to a monocyclic carbocyclic radical, and to a mono-, bi- or tri-cyclic non-aromatic radical containing one or more heteroatoms selected from S, N and O which is mono-bridged or multiply-bridged. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

As used herein the term “ester” refers to a group of formula —COOR, wherein R is a radical actually or notionally derived from the hydroxyl compound ROH.

As used herein the term “amido” refers to a group of formula —CONRₐR_{b}, wherein —NRₐR_{b} is an amino (including cyclic amino) group actually or notionally derived from ammonia or the amine HNRₐR_{b}.

Unless otherwise specified in the context in which it occurs, the term “substituted” as applied to any moiety herein means substituted with at least one substituent, for example selected from (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, halo (including fluoro and chloro), trifluoromethyl, trifluoromethoxy, nitro, nitrile (—CN), oxo, phenyl, —COOH, —COOR^{A}, —COR^{A}, —SO₂R^{A}, —CONH₂, —SO₂NH₂, —CONHR^{A}, —SO₂NHR^{A}, —CONR^{A}R^{B}, SO₂NR^{A}R^{B}, —NH₂, —NHR^{A}, —NR^{A}R^{B}, —OCONH₂, —OCONHR^{A}, —OCONR^{A}R^{B}, —NHCOR^{A}, —NHCOOR^{A}, —NR^{B}COOR^{A}, —NHSO₂OR^{A}, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl group. An “optional substituent” may be one of the foregoing substituent groups.

As used herein the term “salt” includes base addition, acid addition and quaternary salts. Compounds of the invention which are acidic can form salts, including pharmaceutically or veterinarily acceptable salts, with bases such as alkali metal hydroxides, e.g. sodium and potassium hydroxides; alkaline earth metal hydroxides e.g. calcium, barium and magnesium hydroxides; with organic bases e.g. N-ethyl piperidine, dibenzylamine and the like. Those compounds (I) which are basic can form salts, including pharmaceutically or veterinarily acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic and p-toluene sulphonic acids and the like.

For a review on suitable salts, see Handbook of Pharmaceutical Salts: Properties, Selection, and Use by Stahl and Wermuth (Wiley-VCH, Weinheim, Germany, 2002).

The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Compounds with which the invention is concerned which may exist in one or more stereoisomeric form, because of the presence of asymmetric atoms or rotational restrictions, can exist as a number of stereoisomers with R or S stereochemistry at each chiral centre or as atropisomeres with R or S stereochemistry at each chiral axis. The invention includes all such enantiomers and diastereoisomers and mixtures thereof.

So-called ‘pro-drugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

Also included within the scope of the invention are metabolites of compounds of formula (I), that is, compounds formed in vivo upon administration of the drug. Some examples of metabolites include

(i) where the compound of formula (I) contains a methyl group, an hydroxymethyl derivative thereof (—CH₃—>—CH₂OH):
(ii) where the compound of formula (I) contains an alkoxy group, an hydroxy derivative thereof (—OR—>—OH);
(iii) where the compound of formula (I) contains a tertiary amino group, a secondary amino derivative thereof (—NR¹R²—>—NHR¹ or —NHR²);
(iv) where the compound of formula (I) contains a secondary amino group, a primary derivative thereof (—NHR¹—>—NH₂);
(v) where the compound of formula (I) contains a phenyl moiety, a phenol derivative thereof (-Ph->-PhOH); and
(vi) where the compound of formula (I) contains an amide group, a carboxylic acid derivative thereof (—CONH₂—>COOH).

### The Radical R₁

As stated, R₁ is a group of formula -(Alk¹)ₘ-(NH)ₚ-(Alk²)ₙ-Q wherein m, n and p are independently 0 or 1, Alk¹ and Alk² are straight or branched chain divalent C₁-C₆ alkylene or C₂-C₆ alkenylene radicals, and Q is (i) hydrogen, except in the case where m, n and p are both 0, or (ii) an optionally substituted carbocyclic or heterocyclic group.

Q may be, for example, hydrogen, or a cyclopropyl, cyclobutyl, cyclopentyl, cyclohexyl, phenyl, N-piperidinyl, N-piperazinyl, N-morpholinyl, pyridyl, thienyl, furanyl or pyrrolyl ring. It is preferred that Q is hydrogen or a cyclopropyl, cyclobutyl, cyclopentyl, or cyclohexyl ring.

When present in the radical R₁, Alk¹ and Alk² may be, for example, divalent radicals selected from methylene, ethylene, propylene, butylene, ethenylene and propenylene.

In a subclass of compounds with which the invention is concerned, m is 0, p and n are both 1, and Q is hydrogen, so that R₁ is alkylamino. In such cases, -(Alk²)ₙ-Q may be, for example, methyl, ethyl, propyl, isopropyl or tertiary-butyl. Preferred are compounds wherein -(Alk²)ₙ-Q is ethyl, propyl, isopropyl or tertiary-butyl.

In other structures, each of m, p and n is 1, and Q is hydrogen, so that R₁ is alkylaminoalkyl. In such cases, -(Alk²)ₙ-Q may be, for example, methyl, ethyl, propyl, isopropyl or tertiary-butyl, and Alk¹ may be straight or branched chain divalent C₁-C₆ alkylene or C₂-C₆ alkenylene. Preferred are compounds wherein -(Alk²)ₙ-Q is methyl and Alk¹ is C₁-C₄ alkylene, preferably, —CH₂—, —CH₂CH₂—, —CH(CH₃)CH₂—, or —C(CH₃)₂CH₂—.

In other structures, each of m, p and n is 0, and Q is an optionally substituted carbocyclic or heterocyclic group. In such cases, Q may be a cyclopropyl, cyclobutyl, cyclopentyl, cyclohexyl, phenyl, N-piperidinyl, N-piperazinyl, N-morpholinyl, pyridyl, thienyl, furanyl or pyrrolyl ring. It is preferred that Q is a cyclopropyl, cyclobutyl, cyclopentyl, cyclohexyl or pyridyl ring. Particularly preferred are those compounds wherein Q is cyclohexyl or pyridyl.

In other structures, p is 0, each of m and n is 1, and Q is hydrogen or an optionally substituted carbocyclic or heterocyclic group. In such cases, Alk¹ and Alk² are joined directly to form a straight or branched divalent C₁-C₆ alkylene or C₂-C₆ alkenylene chain, terminated by Q. Preferred are compounds wherein -Alk¹-Alk²- is C₁-C₄ alkylene, and Q is hydrogen or [1,2,4]triazol-1-yl. Particularly preferred are those compounds wherein -Alk¹-Alk²-Q is tertiary-butyl.

Preferred are those compounds wherein R₁ is —C(CH₃)₃ or —NHC(CH₃)₃, particularly preferred compounds being those wherein R₁ is —C(CH₃)₃.

### The Group R₂

R₂ is hydrogen, C₁-C₃ alkyl, cyclopropyl, or —CF₃. It is preferred that R₂ is hydrogen or methyl. Particularly preferred compounds are those wherein R₂ is hydrogen.

### The Group Ar

Ar is an optionally substituted phenyl or 5- or 6-membered heteroaryl ring, where the optional substituents are selected from chloro, fluoro, bromo, methyl, trifluoromethyl, methoxy, trifluoromethoxy, cyano, amido, ester, phenyl, pyridyl, or pyrimidinyl. Ar may be, for example, phenyl or pyridyl which is substituted in the 4-position of the ring relative to the ring's point of attachment to the piperazine nitrogen. Preferred are compounds wherein Ar is phenyl substituted in the 4 position by chloro, fluoro, bromo, methyl, trifluoromethyl, or cyano, and additionally optionally substituted in the 2-position by chloro, fluoro, bromo, methyl, trifluoromethyl, methoxy, trifluoromethoxy, cyano, amido, or a C₁-C₅ alkoxycarbonyl group such as methoxycarbonyl or ethoxycarbonyl. Another preferred subset of compounds are those wherein Ar is phenyl substituted in the 4-position by chloro, fluoro, bromo, trifluoromethyl, or cyano, and additionally optionally substituted in the 2-position by chloro, fluoro, bromo or methyl. Particularly preferred compounds are those wherein Ar is phenyl substituted in the 4-position by chloro or fluoro, and additionally optionally substituted in the 2-position by chloro, fluoro or methyl.

Specific examples of Ar groups usable in compounds of the invention include those present in the compounds of the Examples herein.

### Ring A

Ring A is an optionally substituted phenyl or 5- or 6-membered heteroaryl ring. Preferred are those compounds wherein ring A is selected from optionally substituted phenyl or pyridyl, and include those compounds wherein A is a phenyl ring, optionally substituted by one or two substituents selected from chloro, fluoro, bromo, methyl, trifluoromethyl, methoxy, trifluoromethoxy, or cyano. Particularly preferred are those compounds wherein ring A is 1,4-phenylene.

### The Group W-(L)ₛ-

In a subclass of compounds with which the invention is concerned s is 1 and L is —CH₂—, —C(═O)—, —NH—, —O—, —S—, —SO—, —SO₂—, —(CH₂)₂—, —CH═CH—, —OCH₂—, —CH(CH₃)—, or —NH—CH₂—, and W is an optionally substituted N-containing heterocyclic ring of 5 to 7 ring atoms. W may be selected from, for example, optionally substituted pyrrolidinyl, piperidinyl, piperazinyl, homopiperazinyl, morpholinyl, pyridinyl or pyrimidinyl. Preferred compounds within this subclass are those wherein L is —CH₂—, —CH(CH₃)—, or —NH—CH₂—. Other preferred compounds are those wherein L is —CH₂—, —CH(CH₃)—, or —NH—CH₂—, and W is pyrrolidin-3-yl, piperidin-3-yl, piperidin-4-yl, piperazin-1-yl or homopiperazin-1-yl. Particularly preferred structures are those wherein L is —CH₂— and W is homopiperazin-1-yl; L is —CH(CH₃)— and W is piperazin-1-yl or homopiperazin-1-yl; or L is —NH—CH₂— and W is pyrrolidin-3-yl, piperidin-3-yl or piperidin-4-yl.

In another subclass of compounds with which the invention is concerned s is 1 and L is —CH₂—, —C(═O)—, —NH—, —O—, —S—, —SO—, —SO₂—, —(CH₂)₂—, —CH═CH—, or —OCH₂—, and W is an optionally substituted N-containing heterocyclic ring of 5 or 6 ring atoms. W may be selected from, for example, optionally substituted pyrrolidinyl, piperidinyl, piperazinyl, morpholinyl, pyridinyl or pyrimidinyl. Preferred compounds within this subclass are those wherein L is —CH₂—, —C(═O)—, or —NH—. Other preferred compounds are those wherein L is —CH₂—, —C(═O)—, or —NH—, and W is 3-(dimethylamino)-pyrrolidin-1-yl, piperidin-4-yl, 4,4-difluoro-piperidin-1-yl, piperazin-1-yl, 1-methyl-piperazin-4-yl, 1-(tertiarybutyloxycarbonyl)-piperazin-4-yl, 1-(pyridin-4-yl)-piperazin-4-yl, 2-isopropyl-piperazin-1-yl, 2-methyl-piperazin-1-yl, 3-methyl-piperazin-1-yl, 2,6-dimethyl-piperazin-1-yl, 3,5-dimethyl-piperazin-1-yl, 2,5-dimethyl-piperazin-1-yl, morpholin-4-yl, pyridin-4-yl, pyridin-3-yl, 3-methyl-pyridin-4-yl, 2-methyl-pyridin-3-yl, or 2-chloro-pyridin-5-yl. Particularly preferred structures are those wherein L is —CH₂— and W is 3-(dimethylamino)-pyrrolidin-1-yl, piperidin-4-yl, 4,4-difluoro-piperidin-1-yl, piperazin-1-yl, 2-isopropyl-piperazin-1-yl, 2-methyl-piperazin-1-yl, 3-methyl-piperazin-1-yl, 2,6-dimethyl-piperazin-1-yl, 3,5-dimethyl-piperazin-1-yl, 2,5-dimethyl-piperazin-1-yl, morpholin-4-yl, or 2-chloro-pyridin-5-yl; L is —C(═O)— and W is piperidin-4-yl, piperazin-1-yl, 3-methyl-piperazin-1-yl, pyridin-4-yl, pyridin-3-yl, or 2-methyl-pyridin-3-yl; or L is —NH— and W is pyridin-4-yl.

In yet another subclass of compounds with which the invention is concerned s is 0 and W is an optionally substituted N-containing saturated heterocyclic ring of 5 to 7 ring atoms.

In yet another subclass of compounds with which the invention is concerned s is 0 and W is an optionally substituted N-containing saturated heterocyclic ring of 5 or 6 ring atoms. W may be selected from, for example, optionally substituted pyrrolidinyl, piperidinyl, piperazinyl or morpholinyl. Preferred compounds are those wherein W is 3-(dimethylamino)-pyrrolidin-1-yl, piperidin-4-yl, 4,4-difluoro-piperidin-1-yl, piperazin-1-yl, 1-methyl-piperazin-4-yl, 1-(tertiarybutyloxycarbonyl)-piperazin-4-yl, 1-(pyridin-4-yl)-piperazin-4-yl, 2-isopropyl-piperazin-1-yl, 2-methyl-piperazin-1-yl, 3-methyl-piperazin-1-yl, 2,6-dimethyl-piperazin-1-yl, 3,5-dimethyl-piperazin-1-yl, 2,5-dimethyl-piperazin-1-yl, or morpholin-4-yl.

In a further subclass of compounds with which the invention is concerned s is 0 and W is an N-containing heteroaryl ring of 5 or 6 ring atoms substituted by at least one substituent selected from amino, C₁-C₆ alkylamino or cyano. W may be pyridyl or pyrimidinyl, for example, each substituted by one amino, C₁-C₆ alkylamino or cyano group. Preferred compounds include those wherein the pyridyl or pyrimidinyl ring is ortho-substituted relative to its point of attachment, and wherein W is additionally optionally substituted by halo, C₁-C₆ alkyl, C₁-C₆ alkoxy, or halo C₁-C₆ alkyl. Particularly preferred compounds are those wherein W is 2-amino-pyridin-3-yl, 2-cyano-pyridin-3-yl, 3-amino-pyridin-4-yl, 3-cyano-pyridin-4-yl, 2-amino-4-chloro-pyrimidin-6-yl, 2-amino-4-methyl-pyrimidin-6-yl, or 4-amino-6-methyl-pyrimidin-5-yl.

Specific compounds with which the invention is concerned include those of the Examples.

According to a further aspect of the invention, there is provided for use in therapy a compound of formula (I).

According to a further aspect of the invention, there is provided the use of a compound of formula (I) in the manufacture of a medicament for the treatment of a disorder mediated by CB₁ receptors.

According to a further aspect of the present invention there is provided a method of treatment of a disorder mediated by CB₁ receptors comprising administration to a subject in need of such treatment an effective dose of the compound of formula (I), or a pharmaceutically acceptable salt or prodrug thereof.

The disorders mediated by CB₁ receptors are selected from psychosis, memory deficit, cognitive disorders, attention deficit disorder, migraine, neuropathy, neuro-inflammatory disorders including multiple sclerosis and Guillain-Barre syndrome and the inflammatory sequelae of viral encephalitis, cerebral vascular injuries, head trauma, anxiety disorders, depression, stress, epilepsy, dementia, distonia, Alzheimer's disease, Huntingdon's disease, Tourette's syndrome, ischaemia, pain, Parkinson's disease, schizophrenia, substance abuse disorders especially relating to nicotine, alcohol, and opiates, smoking cessation, treatment of nicotine dependence and/or treatment of symptoms of nicotine withdrawal, gastrointestinal disorders (such as dysfunction of gastrointestinal motility or diarrhoea), obesity and other eating disorders associated with excessive food intake, and associated health complications including non-insulin dependant diabetes mellitus.

The present invention is particularly directed to psychosis, memory deficit, cognitive disorders, attention deficit disorder, migraine, anxiety disorders, stress, epilepsy, Parkinson's disease, schizophrenia, substance abuse disorders especially relating to nicotine, alcohol, and opiates, smoking cessation, treatment of nicotine dependence and/or treatment of symptoms of nicotine withdrawal, gastrointestinal disorders (such as dysfunction of gastrointestinal motility or diarrhoea), obesity and other eating disorders associated with excessive food intake, and associated health complications including non-insulin dependant diabetes mellitus.

The present invention is more particularly directed to disorders selected from psychosis, schizophrenia, cognitive disorders, attention deficit disorder, smoking cessation, gastrointestinal disorders (such as dysfunction of gastrointestinal motility or diarrhoea), obesity and other eating disorders associated with excessive food intake (including bulimia and compulsive eating disorder) in juvenile, adolescent and adult patients, and the associated health complications including non-insulin dependant diabetes mellitus. The present invention is particularly directed to obesity and other eating disorders associated with excessive food intake and associated health complications including non-insulin dependant diabetes mellitus, and particularly to obesity and other eating disorders associated with excessive food intake, and especially to obesity.

In an alternative embodiment, the present invention is directed to substance abuse disorders especially relating to nicotine, alcohol, and opiates, smoking cessation, treatment of nicotine dependence and/or treatment of symptoms of nicotine withdrawal, and particularly to smoking cessation and the facilitation thereof.

In a further alternative embodiment, the present invention is directed to gastrointestinal disorders (such as dysfunction of gastrointestinal motility or diarrhoea).

In a further alternative embodiment, the present invention is directed to the treatment of Parkinson's Disease.

In a further alternative embodiment, the present invention is directed to the treatment of bone resorption, osteoporosis, bone cancer or Paget's disease of bone.

The present invention may be employed in respect of a human or animal subject, more preferably a mammal, more preferably a human subject.

As used herein, the term “treatment” as used herein includes prophylactic treatment.

The compound of formula (I) may be used in combination with one or more additional drugs useful in the treatment of the disorders mentioned above, the components being in the same formulation or in separate formulations for administration simultaneously or sequentially.

It will be understood that the specific dose level for any particular patient will depend upon a variety of factors including the activity of the specific compound employed, the age, body weight, general health, sex, diet, time of administration, route of administration, rate of excretion, drug combination and the causative mechanism and severity of the particular disease undergoing therapy. In general, a suitable dose for orally administrable formulations will usually be in the range of 0.1 to 3000 mg, once, twice or three times per day, or the equivalent daily amount administered by infusion or other routes. However, optimum dose levels and frequency of dosing will be determined by clinical trials as is conventional in the art.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

There are multiple synthetic strategies for the synthesis of the compounds (I) with which the present invention is concerned, but all rely on known chemistry, known to the synthetic organic chemist. Thus, compounds according to formula (I) can be synthesised according to procedures described in the standard literature and are well-known to the one skilled in the art. Typical literature sources are “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky), review articles such as found in “Synthesis”, “Acc. Chem. Res.”, “Chem. Rev”, or primary literature sources identified by standard literature searches online or from secondary sources such as “Chemical Abstracts” or “Beilstein”. Such literature methods include those of the preparative Examples herein, and methods analogous thereto.

For example, the following general reaction scheme can be employed:

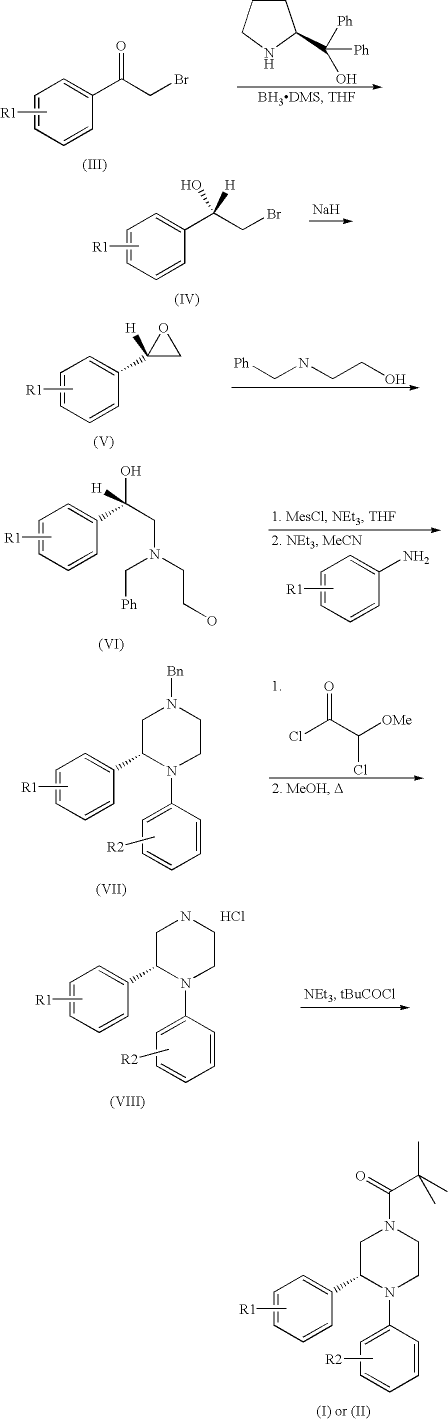

The stereoselective reduction of a suitably substituted acetophenone (III) can be accomplished with standard metal hydride reagents such as borane or lithium aluminium hydride, in the presence of chiral auxiliaries. A large number of chiral auxiliaries have been developed for this type of reaction, for example (R)- or (S)-alpha, alpha diphenyl hydroxymethylpyrrolidine as described in Prasad et al (Tetrahedron:Asymmetery (7), 3147, 1996 and Tetrahedron:Asymmetery (13), 1347, 2002). Alternatively the stereoselective reduction could be completed by transition metal catalysed hydrogenation using hydrogen and a chiral ligand, for example (Noyori, Ryoji et al (Ajinomoto Co., Inc., Japan). PCT Int. Appl. (2002) WO2002051781 A1 20020704). The use of these reagents gives the desired intermediates (IV) stereoselectively. The preferred compounds have S-stereochemistry.

The intramolecular displacement of the alpha-halide (IV) to give the desired suitably substituted chiral styrene oxides (V) can be accomplished with a number of non-nucleophilic bases such as for example potassium carbonate, sodium hydride, sodium hydroxide, potassium tertiary-butoxide or diazabicyclo-undecane (DBU). The preferred stereochemistry is S at the benzylic carbon.

The reaction of a secondary amine with the suitably substituted styrene oxide under thermal conditions to yield the desired hydroxyethylamine is carried out with N-benzylethanolamine to yield the di(hydroxyethyl)amine (VI). The preferred stereochemistry is S at the benzylic carbon.

The transformation of the diol (VI) into an intermediate with two electrophilic centres without racemisation allows the formation of the piperazine core. A number of processes are available for the first part of this transformation including formation of sulfonate esters, for example methane sulfonates or para-toluene sulfonates; or the formation of dihalides either directly using for example thionyl chloride or in two steps from disulfonate intermediates using sodium bromide under Finkelstein conditions. The dielectrophile is then reacted with a primary amine under thermal conditions to yield the desired piperazine, with an inversion of the stereochemistry found in the starting material. In the examples given herein the amines are generally suitably substituted anilines to give the desired 1,2-diaryl-4-benzylpiperazines (VII). The preferred stereochemistry is R as given in 1, 2(R)-diaryl-4-benzylpiperazines.

The deprotection of the benzylamine can be completed either by hydrogenation using a catalyst like palladium on charcoal or platinum oxide, or by carbonylation of the nitrogen followed by nucleophilic attack at the benzylic carbon. This latter transformation can be completed with alkyl chloroformates to yield intermediate carbonates and benzyl chloride. The resultant carbonate can be reacted further under hydrolytic conditions, for example with nucleophilic alcohols, sodium hydroxide and water, to give the free amines. Using 1-chloroethyl chloroformate for this deprotection step gives the 1-chloroethylformyl carbonate, which can be reacted further with methanol under thermal conditions to yield the 1,2-diaryl piperazine (VIII) as the hydrochloride salt. The preferred stereochemistry is R as given in 1, 2(R)-diaryl piperazines.

The organic acids coupled with the piperazine free amine intermediate can be activated using a number of reagents such as isobutyl chloroformate, PyBOP, HATU and hydroxyl-benzotriazole or N-hydroxysuccinimide. Organic acids can also be activated by transformation to the carbonyl chloride, by reaction with for example thionyl chloride or phosphorous oxychloride. Some carbonyl chlorides can be obtained commercially, for example 2,2-dimethylpropionyl chloride (pivalyl chloride). This gives some of the compounds exemplified such as [3-(R)-(aryl)-4-(aryl)-piperazin-1-yl]-2,2-dimethyl-propan-1-one. Obviously this could be modified to a coupling reaction with a number of electrophiles to yield compound (I) or (II) that fit the general formula. For example the 1,2-diaryl piperazine (VIII) could be reacted with isocyanates or carbamates to give ureas, for example N-alkyl-[3-(R)-(aryl)-4-(aryl)-piperazine-1-carboxamides.

Compounds of formula (I), (II), (VII) or (VIII) containing suitable aromatic substituents could be reacted further as shown in the scheme below. For example, compounds of general formula (I) or (II) that contain bromide, iodide or trifluoromethane sulfonate substituents could be reacted under transition metal catalysed couplings to a number of reagents. These reagents could include aryl boronic acids (Suzuki-Miyaura coupling), aryl metal reagents (e.g. aryl zinc, aryl copper or aryl magnesium reagents—Negishi or Sonogashira coupling or aryl tin reagents for Stille coupling), amongst other reagents to give products within the general formula of (I).

Likewise, compounds of formula (I), (II), (VII) or (VIII) containing bromide or iodide substituents could be transformed into organo-metal reagents for further transformations. For example, the bromide or iodide substituent could be transformed into organo-boronates, lithium, magnesium, iron, copper, zinc or tin species for further transformations. These transformations could include transition metal catalysed couplings. This approach would yield compounds of general formula (I) or (II) where R5=alkyl, unsaturated alkyl, aryl, heteroaryl, arylalkyl, arylethylene, or arylalkynyl.

Similarly, nucleophiles can be reacted with compounds of formula (I), (II), (VII) or (VIII) containing suitable aromatic substituents either by direct aromatic nucleophilic substitution or by transition metal catalysed coupling. For example, compounds of formula (I), (II), (VII) or (VIII) containing bromides, iodides or trifluoromethane sulfonate substituents can be coupled with alcohols, amines and amides using palladium catalysed couplings as described by Buchwald or Hartwig to give 3-(R)-(aryl)-4-(aryl)-piperazines where R5 is alkoxy, aryloxy, alkylamino, arylamino, or arylalkylamino.

Equally, the bromide, iodide or trifluoromethane sulfonate substituents can be transformed into reactive centres for further transformations. This could include transformation of bromides, iodides or trifluoromethane sulfonates into compounds of general formula (I) or (II) where R5 is carbaldehyde, carbamide, carboxylic acid or ester. Carbaldehydes can undergo further transformation by for example reductive amination into compounds of general formula (I) or (II) where R5 is for example alkylaminomethyl, arylaminomethyl or arylalkylaminomethyl.

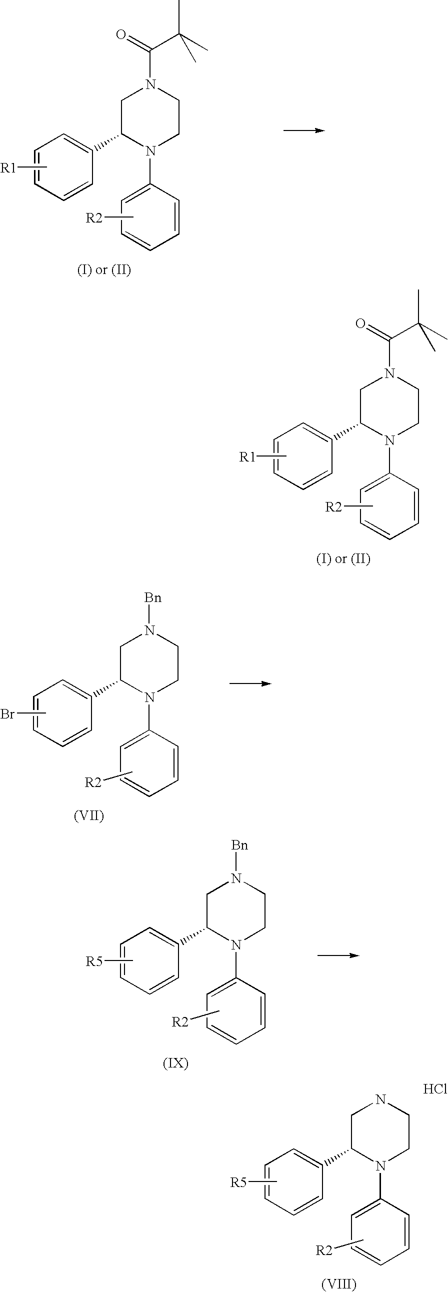

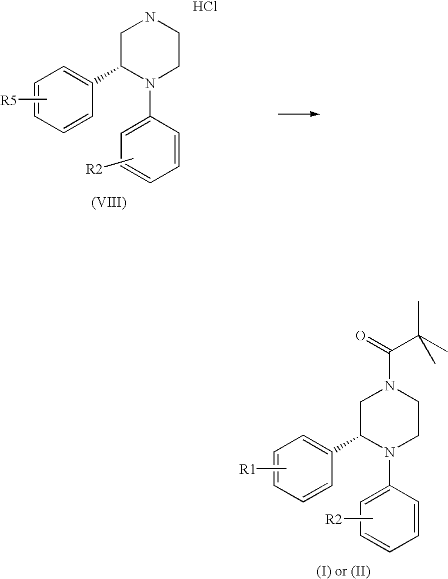

The following examples illustrate the preparation and activities of specific compounds of the invention.

### Preparative Example 1

### Procedure A

### 2-Bromo-1-S-(4-bromo-phenyl)-ethanol

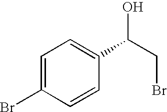

To a solution of borane-dimethylsulphide complex (11.7 mL) under nitrogen was added a solution of (S)-diphenyl prolinol (0.46 g) in THF (30 mL). The mixture was then stirred at 45° C. for 12 hours. To this was then added over a one hour period, 2,4′-dibromoacetophenone, (10 g) using a syringe pump. The mixture was then stirred at 45° C. for one hour and then at room temperature for a further 2 hours. The mixture was cooled to 0° C. and treated with methanol until no more effervescence was seen. The solvents were then removed in vacuo to yield an off white solid. This was taken up in diethyl ether (200 mL) and washed with dilute hydrochloric acid followed by brine. The solvents were dried with sodium sulphate and concentrated in vacuo yielding the 2-Bromo-1-S-(4-bromo-phenyl)-ethanol (9.9 g) that was used without further purification.

LC/MS (method A) retention time 2.43 min, no molecular ion seen. ¹NMR (CDCl₃) δ: 7.51-7.48 (d, 2H, J=10); 7.27-7.25 (d, 2H, J=10 Hz); 4.89-4.87 (dd, 1H, J=10, 3 Hz); 3.62-3.58 (2H, dd, J=10.5, 2.5); 3.51-3.46 (2H, dd, J=10, 9)

### Preparative Example 2

### Procedure B

### 2-S-(4-Bromo-phenyl)-oxirane

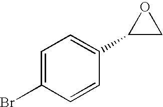

2-Bromo-1-S-(4-bromo-phenyl)-ethanol (9.9 g) was dissolved in THF (70 mL) and cooled to 0° C. To this was added sodium hydride (1.7 g) portion wise over a five minute period. The reaction mixture was then warmed to room temperature and stirred for two hours. After this time TLC indicated complete consumption of starting material. The reaction mixture was cooled to 0° C. and treated carefully with methanol and then the reaction mixture reduced to dryness. Dichloromethane (70 mL) was then added and washed with water, brine and dried over sodium sulphate to give the 2-S-(4-Bromo-phenyl)-oxirane (7.7 g) that was used without further purification.

LC/MS (method A) retention time 2.53 min. no molecular ion seen. ¹NMR (CDCl₃) δ: 7.46-7.43 (d, 2H, J=8); 7.14-7.11 (d, 2H, J=8 Hz); 3.81-3.79 (dd, 1H, J=4, 2); 3.13-3.11 (2H, dd, J=5, 4); 2.73-2.71 (2H, dd, J=5, 3).

The enantiomeric excess of this material was determined to be >96% using NMR and the chiral shift reagent (R)-(−)-□-(trifluoromethyl)anthracene-9-methanol (R-isomer of Pirkle's alcohol) based on a comparison with the racemic material. See “Nuclear magnetic resonance determination of enantiomeric compositions of oxaziridines using chiral solvating agents.” Pirkle, W. H.; Rinaldi, P. L. Journal of Organic Chemistry (1977), 42(20), 3217-19 and references cited therein.

### Preparative Example 3

### Procedure C

### 2-[Benzyl-(2-S-hydroxy-ethyl)-amino]-1-(4-bromo-phenyl)-ethanol

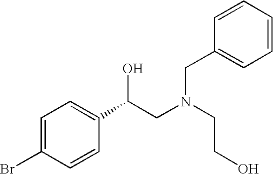

2-S-(4-Bromo-phenyl)-oxirane (7.3 g) and N-benzylethanolamine (7.8 mL) were heated to 130° C. for 12 hours. After this time the solution was cooled to room temperature and applied directly to a silica column using ethyl acetate-hexanes as eluent. This gave the 2-[Benzyl-(2-S-hydroxy-ethyl)-amino]-1-(4-bromo-phenyl)-ethanol (9.0 g) as a colourless oil.

LC/MS (method A) retention time 1.82 min [M+H]⁺ 350 bromine splitting pattern

### Preparative Example 4

### Procedure D

### 4-Benzyl-2-R-(4-bromo-phenyl)-1-(4-chloro-phenyl)-piperazine

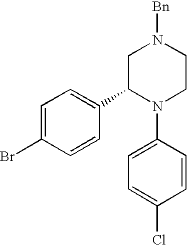

2-[Benzyl-(2-S-hydroxy-ethyl)-amino]-1-(4-bromo-phenyl)-ethanol (9 g) was dissolved in THF (90 mL) and cooled to 0° C. To this was slowly added triethylamine (17.5 mL) followed by methanesulphonyl chloride (5.97 mL). The reaction mixture was then warmed to room temperature and stirring continued for one hour. After this time, the reaction was concentrated in vacuo and acetonitrile (90 mL) added followed by 4-chloroaniline (4.9 g) and the reaction mixture brought to reflux for 12 hours. The reaction mixture was then cooled and the solvents evaporated. The oily residue remaining was partitioned between sodium carbonate solution and dichloromethane. The organic layer was then dried and evaporated producing an orange oil which was purified by column chromatography using ethyl acetate-hexanes as eluent. This gave the 4-benzyl-2-R-(4-bromo-phenyl)-1-(4-chloro-phenyl)-piperazine (6.1 g) as a pale yellow oil.

LC/MS (method A) retention time 2.57 min [M+H]⁺ 442 bromine-chlorine splitting pattern

### Preparative Example 5

### Procedure E

### 3-R-(4-Bromo-phenyl)-4-(4-chloro-phenyl)-piperazine-1-carboxylic acid 1-chloro-ethyl ester

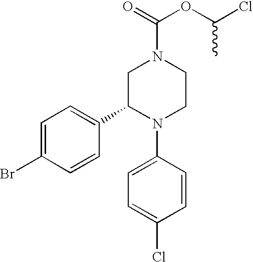

4-Benzyl-2-R-(4-bromo-phenyl)-1-(4-chloro-phenyl)-piperazine (6.1 g) was dissolved in dichloromethane (60 mL) and cooled to 0° C. To this was slowly added 1-chloroethylchloroformate (7.4 mL). The mixture was then stirred at room temperature for 2 hours after which time all starting material had been consumed. The reaction mixture was then re-cooled and quenched with 2N sodium hydroxide solution. The organic layers were then dried and evaporated producing an orange oil which was purified by column chromatography using ethyl acetate-hexanes as eluent. This gave the 3-R-(4-Bromo-phenyl)-4-(4-chloro-phenyl)-piperazine-1-carboxylic acid 1-chloro-ethyl ester (3.9 g) as a pale yellow oil.

LC/MS (method A) retention time 3.02 min [M+H]⁺ 458 bromine-dichlorine splitting pattern

### Preparative Example 6

### Procedure F

### 2-R-(4-Bromo-phenyl)-1-(4-chloro-phenyl)-piperazine hydrochloride

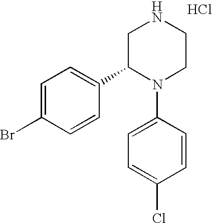

3-R-(4-Bromo-phenyl)-4-(4-chloro-phenyl)-piperazine-1-carboxylic acid 1-chloro-ethyl ester (3.9 g) was dissolved in methanol (40 mL) and heated to reflux for 2 hours. After this time the mixture was allowed to cool and the solvents were evaporated yielding the 2-R-(4-Bromo-phenyl)-1-(4-chloro-phenyl)-piperazine hydrochloride (3.2 g) which was used without further purification.

LC/MS (method A) retention time 1.99 min [M+H]⁺ 351 bromine-chlorine splitting pattern

### Preparative Example 7

### Procedure G—Preparation of Amide or Urea

### Intermediate 1

### 1-[3-R-(4-Bromo-phenyl)-4-(4-chloro-phenyl)-piperazin-1-yl]-2,2-dimethyl-propan-1-one

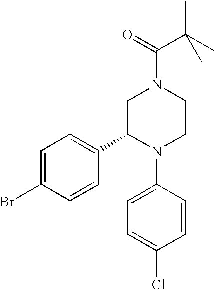

2-R-(4-Bromo-phenyl)-1-(4-chloro-phenyl)-piperazine hydrochloride (2.1 g) was dissolved in DCM (15 mL) and cooled to 0° C. To this was added triethylamine (4.1 mL) followed by pivalyl chloride (0.8 mL). The reaction mixture was slowly allowed to warm to room temperature and stirring continued for 1 hour. After this time all starting material had been consumed. The reaction mixture was washed with sodium bicarbonate solution and the organic layers dried and evaporated producing an orange oil which was purified by column chromatography using ethyl acetate-hexanes as eluent. This gave intermediate 1, 1-[3-R-(4-Bromo-phenyl)-4-(4-chloro-phenyl)-piperazin-1-yl]-2,2-dimethyl-propan-1-one (2.1 g) as a colourless oil.

LC/MS (method A) retention time 2.98 min [M+H]⁺ 435 bromine-chlorine splitting pattern

### Intermediate 2

### 1-[3-R-(4-Bromo-phenyl)-4-(4-chloro-2-fluoro-phenyl)-piperazin-1-yl]-2,2-dimethyl-propan-1-one

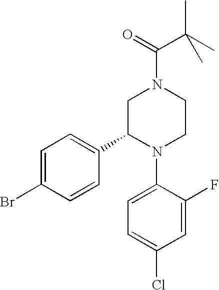

1-[3-R-(4-Bromo-phenyl)-4-(4-chloro-2-fluoro-phenyl)-piperazin-1-yl]-2,2-dimethyl-propan-1-one was prepared in the same way as described for Intermediate 1, using Procedures A-G. However 4-chloro-2-fluoro-aniline was used in Procedure D.

LC/MS (method A) retention time 2.92 min [M+H]+ 455; bromine-chlorine splitting pattern

### Intermediate 3

### 1-[3-R-(4-Bromo-phenyl)-4-(2,4-dichlorophenyl)-piperazin-1-yl]-2,2-dimethyl-propan-1-one

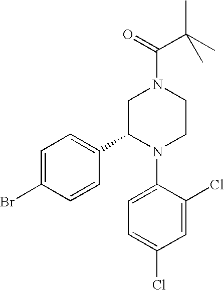

1-[3-R-(4-Bromo-phenyl)-4-(2,4-dichlorophenyl)-piperazin-1-yl]-2,2-dimethyl-propan-1-one was prepared in the same way as described for Intermediate 1, using Procedures A-G. However 2,4-dichloro-aniline was used in Procedure D.

LC/MS (method A) retention time 3.00 min [M+H]+ 469; splitting pattern showing bromine and two chlorines present in molecule.

### Preparative Example 8

### Procedure H

### Intermediate 4

### (R)-1-(4-Chloro-2-fluoro-phenyl)-4-trimethylacetyl-2-[4-(2-(4,4,5,5-tetramethyl[1.3.2]dioxanborolyl]]piperazine

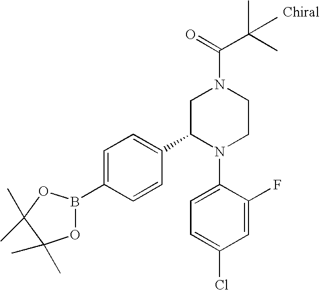

1-[3-R-(4-Bromo-phenyl)-4-(4-chloro-2-fluoro-phenyl)-piperazin-1-yl]-2,2-dimethyl-propan-1-one (88 mg) was dissolved in DME (4 mL), with bis-pinnacolatodiboron (54 mg), [1,1-bis(diphenylphosphino)ferrocene]palladium (II) chloride (1:1 complex with DCM) (8 mg) and potassium acetate (57 mg). The resultant suspension was stirred for 5 minutes and then heated in a microwave reactor at 150° C. for 10 minutes. The reaction mixture was cooled to room temperature, diluted with ethyl acetate (8 mL), concentrated in vacuo on to silica (5 g). The solid was purified by column chromatography (eluant ethyl acetate/isohexane, 2:3 ratio v/v) to yield the desired (R)-1-(4-Chloro-2-fluoro-phenyl)-4-trimethylacetyl-2-[4-(2-(4,4,5,5-tetramethyl[1.3.2]-dioxanborolyl]]piperazine, as a white tacky solid (81 mg).

LC/MS (method C) retention time 4.54 min [M+H]+ 501; chlorine splitting pattern.

### Example 1

### Procedure I—Suzuki Coupling

### 1-{4-(4-Chloro-2-fluoro-phenyl)-3-R-[4-(2-amino-4-chloro-pyrimidin-6-yl)-phenyl]-piperazin-1-yl}-2,2-dimethyl-propan-1-one

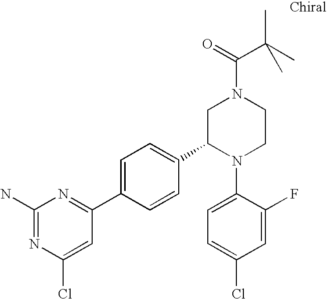

(R)-1-(4-Chloro-2-fluoro-phenyl)-4-trimethylacetyl-2-[4-(2-(4,4,5,5-tetramethyl[1.3.2]dioxanborolyl]]piperazine (41 mg) and 2-amino-4,6-dichloropyrimidine (12 mg), were dissolved in THF (0.5 mL) and treated with potassium carbonate (30 mg) and water (50 uL). To this suspension was added [1,1-bis(diphenylphosphino)ferrocene]palladium (II) chloride (1:1 complex with DCM) (4 mg) and the resultant suspension heated in a microwave reactor at 150° C. for 10 minutes. The reaction mixture was cooled to room temperature, diluted with ethyl acetate (8 mL), concentrated in vacuo on to silica (5 g). The solid was purified by column chromatography (eluant ethyl acetate/isohexane, 1:4 to 2:3 ratio v/v) to yield the desired 1-{4-(4-Chloro-2-fluoro-phenyl)-3-R-[4-(2-amino-4-chloro-pyrimidin-6-yl)-phenyl]-piperazin-1-yl}-2,2-dimethyl-propan-1-one as a white solid (12 mg).

LC/MS (method C) retention time 4.09 min [M+H]+ 503; splitting pattern shows two chlorines.

### Example 2

### Procedure J—Buchwald Coupling

### 1-{4-(4-Chloro-phenyl)-3-R-[4-(4-methyl-piperazin-1-yl)-phenyl]-piperazin-1-yl}-2,2-dimethyl-propan-1-one

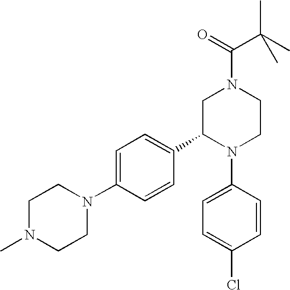

The 1-[3-(4-Bromo-phenyl)-4-(4-chloro-phenyl)-piperazin-1-yl]-2,2-dimethyl-propan-1-one (0.05 g) (example 1), and sodium tert-butoxide (0.015 g) were dissolved in toluene (1.5 mL). To this was added the N-methylpiperazine (0.015 mL), the palladium (II) acetate (approximately 0.001 g) and the 2-(dicyclohexylphosphino)biphenyl (approximately 0.001 g). Nitrogen was bubbled through the reaction mixture for several minutes and then heated to 110° C. for 12 hours. After this time, the reaction mixture was cooled and washed with sodium bicarbonate solution, the aqueous layer extracted with dichloromethane and the organic layers dried and evaporated producing an orange oil which was purified by column chromatography using ethyl acetate-hexanes as eluant. This gave the 1-{4-(4-Chloro-phenyl)-3-R-[4-(4-methyl-piperazin-1-yl)-phenyl]-piperazin-1-yl}-2,2-dimethyl-propan-1-one (0.011 g) as a gum.

LC/MS (method A) retention time 2.13 min [M+H]⁺ 455 chlorine splitting pattern

### Procedure K

### Intermediate 5

### 4-(4-Chloro-phenyl)-3-(4-formyl-phenyl)-piperazine-1-carboxylic acid tert-butyl ester

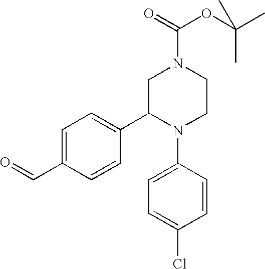

3-(4-Bromo-phenyl)-4-(4-chloro-phenyl)-piperazine-1-carboxylic acid tert-butyl ester (0.25 g) was dissolved in THF (6 mL) and cooled to −78° C. To this was slowly added tBuLi (0.39 mL) and the reaction stirred for 15 minutes at −78° C. Dimethyl formamide (0.16 mL) was then added and the reaction stirred for 50 minutes at room temperature. The reaction mixture was then evaporated and the gum partitioned between water and DCM, the organic layers were washed with water and dried which produced a yellow oil which was purified by column chromatography using ethyl acetate-hexanes as eluent yielding the 4-(4-Chloro-phenyl)-3-(4-formyl-phenyl)-piperazine-1-carboxylic acid tert-butyl ester as a colourless solid (0.041 g).

LC/MS (method A) retention time 2.85 min [M+H]⁺ 442 chlorine splitting pattern

### Example 3

### Procedure L—Reductive Amination

### 4-(4-Chloro-phenyl)-3-(4-diethylaminomethyl-phenyl)-piperazine-1-carboxylic acid tert-butyl ester

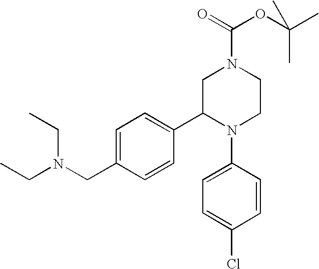

4-(4-Chloro-phenyl)-3-(4-formyl-phenyl)-piperazine-1-carboxylic acid tert-butyl ester (0.01 g) and diethylamine (0.02 mL) were dissolved in dichloroethane (1 mL) and stirred at room temperature of 2 hours. To this was added sodium triacetoxyborohydride (0.042 g) and stirring continued for 12 hours. After this time the reaction was quenched with methanol (1 mL) and the solvents evaporated. The resulting gum was partitioned between water and DCM, the organic layers were washed with water and dried which produced a yellow oil which was purified by preparative HPLC yielding the 4-(4-Chloro-phenyl)-3-(4-diethylaminomethyl-phenyl)-piperazine-1-carboxylic acid tert-butyl ester (example 5), as a colourless solid (0.01 g).

LC/MS (method A) retention time 2.19 min [M+H]⁺ 442 chlorine splitting pattern

N-Boc products from Procedures K and L, like example 3, can be deprotected as described in Procedures P, Q or R. The free amines can then be used to prepare amides or ureas as described in Procedure G.

Procedure K and L can also be applied to N-pivalamide intermediates, such as intermediates 1 to 3.

### Example 4

### Procedure M—Negishi Coupling

### 1-[3-R-(4-(2-chloropyridin-5-yl)methyl)-phenyl)-4-(2,4-dichlorophenyl)-piperazin-1-yl]-2,2-dimethyl-propan-1-one

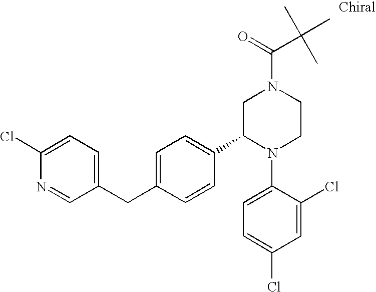

Intermediate 3, 1-[3-R-(4-Bromo-phenyl)-4-(2,4-dichlorophenyl)-piperazin-1-yl]-2,2-dimethyl-propan-1-one (107 mg) was dissolved in THF (5 mL), with tetrakis(triphenylphosphine) palladium (24 mg), under nitrogen. Then (2-chloropyridin-5-yl)methylzinc chloride was added as a solution (0.5M in THF, 0.91 mL) over 2-3 minutes. The resultant solution was then heated in a microwave reactor at 100° C. for 10 minutes and then 120° C. for 10 minutes. The vessel was cooled to room temperature, concentrated in vacuo on to silica (1 g). The solid was purified by column chromatography (eluant ethyl acetate/isohexane, 1:10 v/v ratio) to yield the desired 1-[3-R-(4-(2-chloropyridin-5-yl)methyl)-phenyl)-4-(2,4-dichlorophenyl)-piperazin-1-yl]-2,2-dimethyl-propan-1-one (32 mg) as a white foam.

LC/MS (method C) retention time 4.40 min [M+H]⁺ 518.1 splitting pattern for two chlorines.

### Example 5

### Procedure N

### 1-{4-(2,4-dichlorophenyl)-3-R-[4-(1-piperazinecarboxamide)-phenyl]-piperazin-1-yl}-2,2-dimethyl-propan-1-one

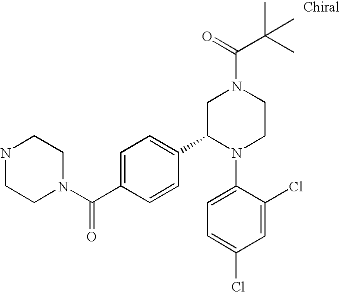

Intermediate 3, 1-[3-R-(4-Bromo-phenyl)-4-(2,4-dichlorophenyl)-piperazin-1-yl]-2,2-dimethyl-propan-1-one (47 mg), 1-benzylpiperazine (22 mg), sodium carbonate (32 mg), trans-di-□-acetato-bis[2-diorthotolylphosphino)benzyl]-dipalladium (II) (5 mg) and molybdenum hexacarbonyl (13 mg) were dissolved in THF (1 mL) and water (0.23 mL). The yellow suspension was then heated in a microwave reactor at 170° C. for 10 minutes. The reaction mixture was then cooled to room temperature, diluted with DCM (10 mL) and concentrated on to silica (2 g). The resultant solid was purified by chromatography (eluant ethyl acetate:isohexane, 2:3 to 1:0 v/v ratio) to give 1-[3-R-(4-(4-benzyl-1-piperazinecarboxamide)-phenyl)-4-(2,4-dichlorophenyl)-piperazin-1-yl]-2,2-dimethyl-propan-1-one (16 mg) as a colourless film.

LC/MS (method C) retention time 7.52 min [M+H]⁺ 593.2 splitting pattern for two chlorines.

1-[3-R-(4-(4-benzyl-1-piperazinecarboxamide)-phenyl)-4-(2,4-dichlorophenyl)-piperazin-1-yl]-2,2-dimethyl-propan-1-one (16 mg) in chloroform (1 mL) was treated with 1-chloroethyl-1-chloroformate (15 mg) and allowed to stand at room temperature for 18 hrs. More 1-chloroethyl-1-chloroformate (30 mg) was added and the reaction allowed to stand at room temperature for a further 4 hrs. The mixture was concentrated in vacuo, re-dissolved in methanol (1 mL) and left standing at room temperature for 60 hrs. The reaction mixture was concentrated in vacuo, dissolved in DCM (10 mL), washed with NaOH (1N, 10 mL). Layers separated and the organic layer concentrated on to silica (2 g). The resultant solid was purified by chromatography (eluant triethylamine:ethanol:ethyl acetate, 2:10:88 to 2:40:58 v/v/v ratios) to give Example 51-[3-R-(4-(1-piperazinecarboxamide)-phenyl)-4-(2,4-dichlorophenyl)-piperazin-1-yl]-2,2-dimethyl-propan-1-one (6 mg) as a colourless film.

LC/MS (method C) retention time 5.90 min [M+H]⁺ 504.7 splitting pattern for two chlorines.

### Example 6

### Procedure O

### 1-{4-(4-Chloro-2-methyl-phenyl)-3-R-[4-(4-pyridinyl-carbonyl)-phenyl]-piperazin-1-yl}-2,2-dimethyl-propan-1-one

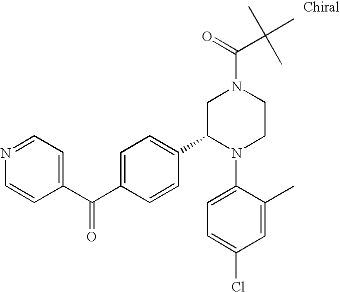

1-{4-(4-Chloro-2-methyl-phenyl)-3-R-[4-dihydroxyboronylphenyl]-piperazin-1-yl}-2,2-dimethyl-propan-1-one was prepared in a similar way to intermediate 4 from 1-{4-(4-Chloro-2-methyl-phenyl)-3-R-[4-bromophenyl]-piperazin-1-yl}-2,2-dimethyl-propan-1-one. 1-{4-(4-Chloro-2-methyl-phenyl)-3-R-[4-dihydroxyboronylphenyl]-piperazin-1-yl}-2,2-dimethyl-propan-1-one (39 mg), nicotinic acid (23 mg), dimethyl dicarbonate (25 mg) and tetrakis(triphenylphosphine) palladium (11 mg) were dissolved in 1,4-dioxane (2 mL) and heated at 100° C., under nitrogen for 18 hrs. The crude reaction mixture was concentrated on to silica (1 g) and purified by column chromatography (eluant 1:1 isohexane:ethyl acetate) to yield the desired 1-{4-(4-Chloro-2-methyl-phenyl)-3-R-[4-(4-pyridinyl-carbonyl)-phenyl]-piperazin-1-yl}-2,2-dimethyl-propan-1-one (28 mg) as a yellow gum.

LC/MS (method B) retention time 4.05 min [M+H]⁺ 476 chlorine splitting pattern.

### Example 7

### Procedure P

### 1-{4-(4-Chloro-2-fluoro-phenyl)-3-R-[4-(1-(homopiperazin-1-yl)-eth-1-yl)-phenyl]-piperazin-1-yl}-2,2-dimethyl-propan-1-one

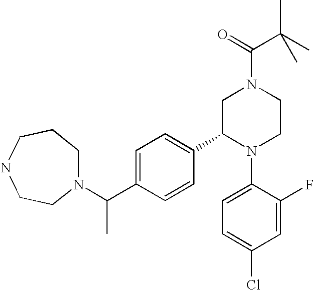

Intermediate 2 was used to make 1-{4-(4-Chloro-2-fluoro-phenyl)-3-R-[4-formyl)-phenyl]-piperazin-1-yl}-2,2-dimethyl-propan-1-one as described for intermediate 5 using Procedure K.

1-{4-(4-Chloro-2-fluoro-phenyl)-3-R-[4-formyl-phenyl]-piperazin-1-yl}-2,2-dimethyl-propan-1-one (250 mg) was dissolved in THF (5 mL) under nitrogen and cooled to 0° C. Methyl magnesium bromide (1.4M in THF/toluene, 0.445 mL) was added over 5 minutes. The reaction was stirred at 0° C. for 15 minutes and then hydrochloric acid (2M, 10 mL) was added dropwise over 5 minutes. The mixture was extracted with DCM. Organic layers were dried (MgSO₄), filtered and concentrated in vacuo. The residue was purified by chromatography (eluant isohexane:ethyl acetate, 1:1, v/v ratio) to yield the desired 1-{4-(4-Chloro-2-fluoro-phenyl)-3-R-[4-(1-hydroxy-ethan-1-yl)-phenyl]-piperazin-1-yl}-2,2-dimethyl-propan-1-one (131 mg) as a white solid.

LC/MS (method A) retention time 2.59 min [M+H]⁺ 419.2 chlorine splitting pattern.

1-{4-(4-Chloro-2-fluoro-phenyl)-3-R-[4-(1-hydroxy-ethan-1-yl)-phenyl]-piperazin-1-yl}-2,2-dimethyl-propan-1-one (81 mg) was dissolved in DCM (3 mL). Carbon tetrabromide (96 mg) and polymer supported triphenyl phosphine (242 mg) were added and the resultant mixture stirred at room temperature for 1 hr. The reaction mixture was filtered, washing polymer with DCM and the collected washes were concentrated in vacuo. The residue was suspended in acetonitrile (2 mL) and 1-Boc-homopiperazine (113 uL) added dropwise. The reaction was stirred at room temperature for 1 hr. The mixture was diluted with DCM (10 mL) and water (10 mL). The layers were separated, organic layer dried (MgSO₄), filtered and concentrated in vacuo. The residue was purified by chromatography (eluant hexane to hexane:ethyl acetate 1:1 v/v ratio) to yield the desired intermediate 1-{4-(4-Chloro-2-fluoro-phenyl)-3-R-[4-(1-(4-butoxycarbonyl-homopiperazin-1-yl)-ethan-1-yl)-phenyl]-piperazin-1-yl}-2,2-dimethyl-propan-1-one.

LC/MS (method A) retention time 2.15 min [M+H]⁺ 601.3 chlorine splitting

1-{4-(4-Chloro-2-fluoro-phenyl)-3-R-[4-(1-(4-butoxycarbonyl-homopiperazin-1-yl)-ethan-1-yl)-phenyl]-piperazin-1-yl}-2,2-dimethyl-propan-1-one was dissolved in DCM (3 mL) and trifluoroacetic acid added in one portion (0.5 mL). The mixture was stirred at room temperature for 1 hr and NaOH (2M, 20 mL) added dropwise. The mixture was extracted with DCM. Organic layers were washed with NaOH (2M), dried (MgSO₄), filtered and concentrated in vacuo. The residue was purified by chromatography (eluant methanol:DCM 1:4 v/v ratio, then the column flushed with ammonia/methanol/DCM 1:5:10 v/v/v ratio) to yield the desired example 71-{4-(4-Chloro-2-fluoro-phenyl)-3-R-[4-(1-(homopiperazin-1-yl)-ethan-1-yl)-phenyl]-piperazin-1-yl}-2,2-dimethyl-propan-1-one as a white solid (22 mg).

LC/MS (method A) retention time 1.80 min [M+H]⁺ 501.3 chlorine splitting pattern.

### Example 8

### Procedure Q

### 1-{4-(4-Chloro-2-fluoro-phenyl)-3-R-[4-(4-piperidinoyl)-phenyl]-piperazin-1-yl}-2,2-dimethyl-propan-1-one

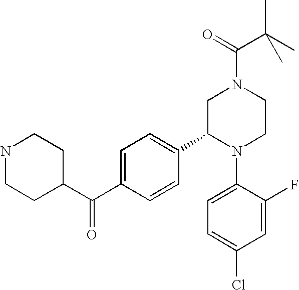

Intermediate 2 (167 mg) was dissolved in THF (2 mL) and cooled to −78° C., then nBuLi (2.5M in hexanes, 0.176 mL) was added dropwise over 5 mins. The reaction was stirred at −78° C. for 30 minutes, and N-Boc-isonipecotic N-methyl-O-methyl hydroxamide (120 mg) was added dropwise as a solution in THF (2 mL). The reaction was kept at −78° C. for 10 minutes and then allowed to warm to room temperature over the next 18 hrs. Water was added, the mixture diluted with NaHCO3 (sat. aq. solution, 10 mL) and extracted with DCM. DCM layers were washed with water, brine, dried (Na₂SO₄), filtered and concentrated in vacuo. The residue was purified by chromatography (eluant ethyl acetate:isohexane 2:3 v/v ratio) to yield the desired intermediate 1-{4-(4-Chloro-2-fluoro-phenyl)-3-R-[4-(1-butoxycarbonyl-4-piperidinoyl)-phenyl]-piperazin-1-yl}-2,2-dimethyl-propan-1-one (50 mg).

This material was deprotected using TFA/DCM, as described for example 7 (Procedure P), to yield the desired 1-{4-(4-Chloro-2-fluoro-phenyl)-3-R-[4-(4-piperidinoyl)-phenyl]-piperazin-1-yl}-2,2-dimethyl-propan-1-one

LC/MS (method A) retention time 1.96 min [M+H]⁺ 486.2; chlorine splitting pattern.

### Example 9

### Procedure R

### 1-{4-(4-Chloro-2-fluoro-phenyl)-3-R-[4-((4-piperidine)methyl)-phenyl]-piperazin-1-yl}-2,2-dimethyl-propan-1-one

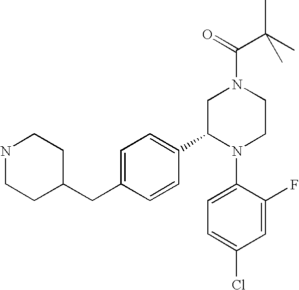

9-BBN (0.5M solution in THF, 0.91 mL) was added to N-Boc-4-methylene-piperidine (96 mg), under nitrogen. The resulting solution was heated at reflux for 90 minutes and allowed to cool to room temperature. DMF (1 mL), water (0.1 mL), [1,1-bis(diphenylphosphino)ferrocene]palladium (II) chloride (1:1 complex with DCM) (11 mg) and intermediate 2,1-[3-R-(4-Bromo-phenyl)-4-(4-chloro-2-fluoro-phenyl)-piperazin-1-yl]-2,2-dimethyl-propan-1-one (189 mg) were added. The resultant mixture was heated to 60° C., under nitrogen, for 18 hours. The reaction was cooled to room temperature, diluted with water and basified with NaOH (2M). The mixture was extracted with DCM, and subsequent DCM layers washed with NaOH (2M), brine, dried (Na₂SO₄), filtered and concentrated in vacuo. The residue was purified by chromatography (eluant ethyl acetate:isohexane 1:4 v/v ratio) to yield 1-{4-(4-Chloro-2-fluoro-phenyl)-3-R-[4-(4-((1-Boc-piperidine)methyl)-phenyl]-piperazin-1-yl}-2,2-dimethyl-propan-1-one (137 mg) as an off white solid.

1-{4-(4-Chloro-2-fluoro-phenyl)-3-R-[4-(4-((1-Boc-piperidine)methyl)-phenyl]-piperazin-1-yl}-2,2-dimethyl-propan-1-one (137 mg) was dissolved in DCM (3 mL) and trifluoroacetic acid (0.4 mL) added in one portion. The reaction was stirred at room temperature for 1 hour. NaOH (2M) was added to basify the reaction mixture and then extracted with DCM. The DCM layer was washed with NaOH (2M), dried (Na₂SO₄), filtered and concentrated in vacuo. The residue was purified by chromatography (eluant ammonia (7N in MeOH):DCM 1:19 v/v ratio) to yield Example 9, 1-{4-(4-Chloro-2-fluoro-phenyl)-3-R-[4-((4-piperidine)methyl)-phenyl]-piperazin-1-yl}-2,2-dimethyl-propan-1-one (50 mg) as an off white solid.

LC/MS (method A) retention time 2.02 min [M+H]⁺ 472.3; chlorine splitting pattern.

The compounds listed in Table 1 were prepared in a similar way to Preparative Examples 1 to 8 and Examples 1 to 9, using alternative reagents where appropriate. The Procedures of Preparative Examples 1 to 8 and Examples 1 to 9 that were employed are indicated in the table.

TABLE 1

| | | LC | | |
|---|---|---|---|---|
| | | retention | Mass | |
| Example | | time | detected | |
| No. | Structure | (mins) | [M + H]+ | Procedures |
| 10 | 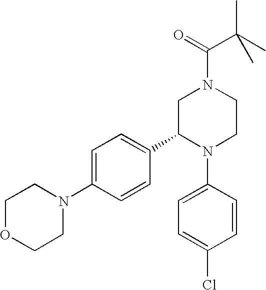 | 2.80* | 442^{a} | A through to G then to J |
| 11 | 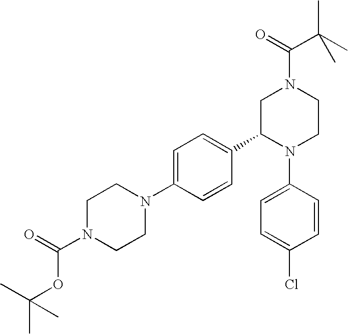 | 2.97* | 541^{a} | A through to G then to J |
| 12 | 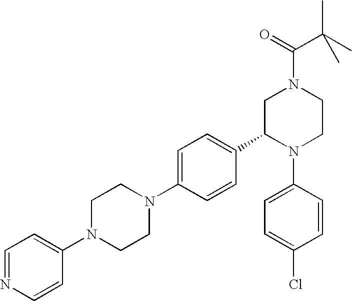 | 2.26* | 518^{a} | A through to G then to J |
| 13 | 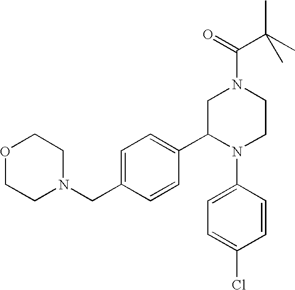 | 2.03* | 456^{a} | A through to G, then to K to L |
| 14 | 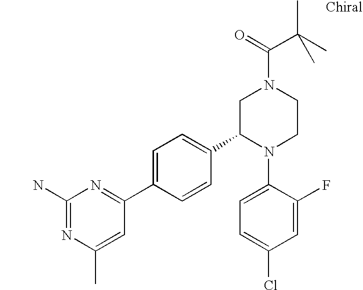 | 5.68^{@} | 482^{a} | A to H then I |
| 15 | 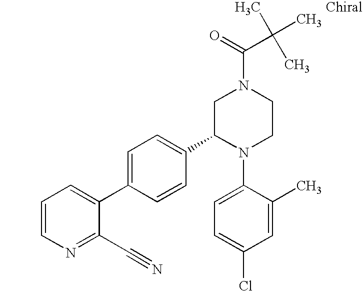 | 4.06^{@} | 473^{a} | A to H then I |
| 16 | 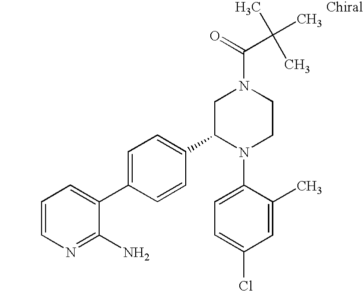 | 3.61^{@} | 463^{a} | A to H then I |
| 17 | 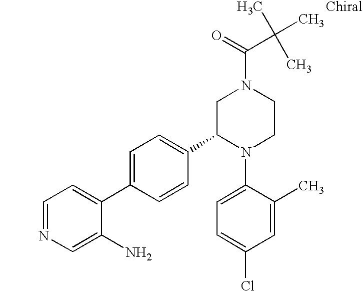 | 3.58^{@} | 463^{a} | A to H then I |
| 18 | 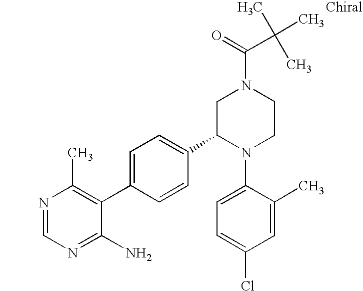 | 3.43^{@} | 478^{a} | A to H then I |
| 19 | 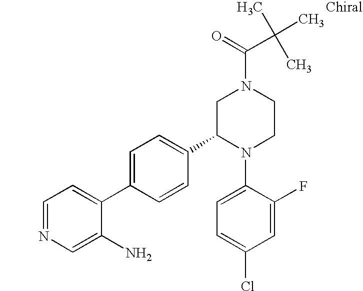 | 3.48^{@} | 467^{a} | A to H then I |
| 20 | 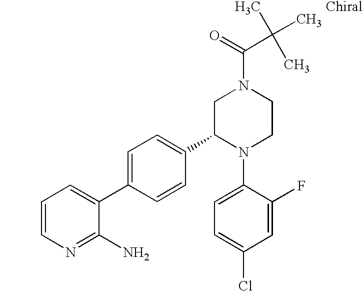 | 3.46^{@} | 467^{a} | A to H then I |
| 21 | 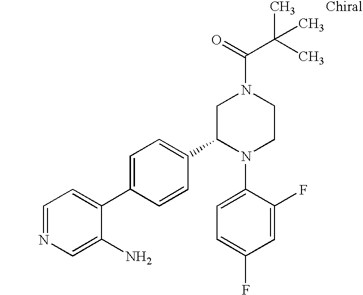 | 2.22^{@} | 450.5 | A to H then I |
| 22 | 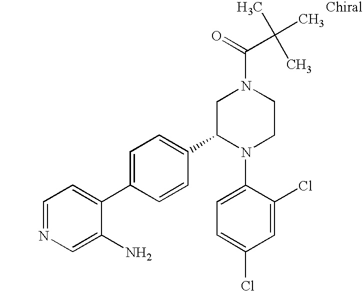 | 3.81^{@} | 483.4^{b} | A to H then I |
| 23 | 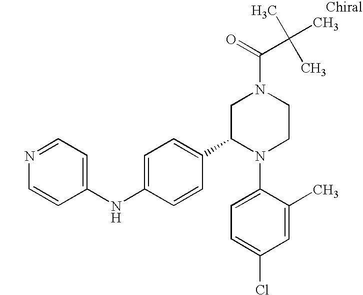 | 5.79^{@} | 463^{a} | A to G then J |
| 24 | 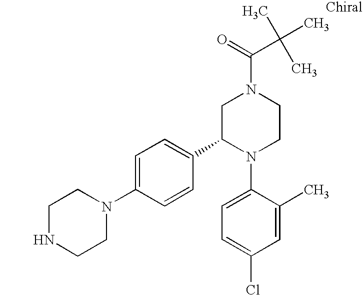 | 3.79* | 455^{a} | A to G then J |
| 25 | 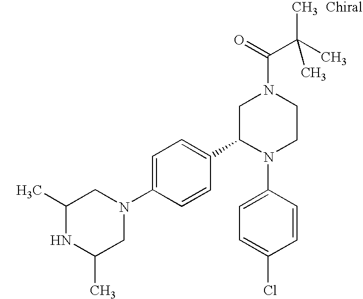 | 7.29* | 469.1^{a} | A to G then J |
| 26 | 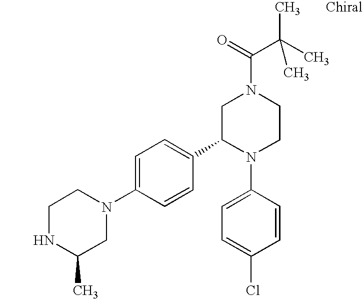 | 5.15* | 455^{a} | A to G then J |
| 27 | 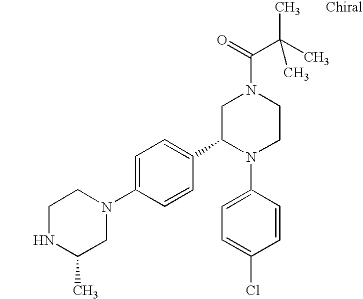 | 9.39* | 455^{a} | A to G then J |
| 28 | 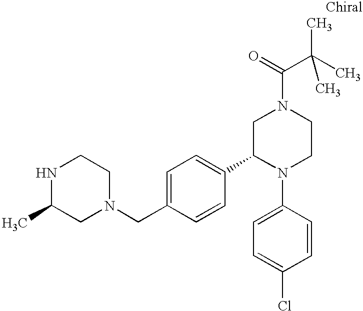 | 3.39* | 469.1^{a} | A to G then K to L |
| 29 | 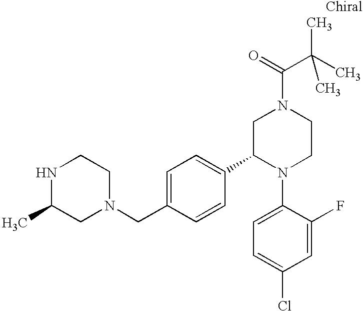 | 3.39* | 487.1^{a} | A to G then K to L |
| 30 | 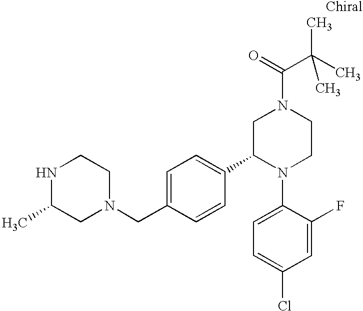 | 1.96* | 487.1^{a} | A to G then K to L |
| 31 | 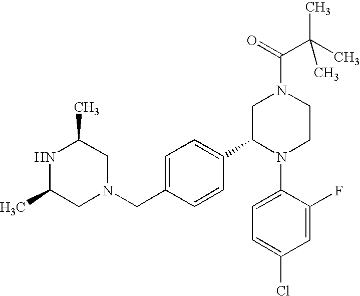 | 1.95* | 501.1^{a} | A to G then K to L |
| 32 | 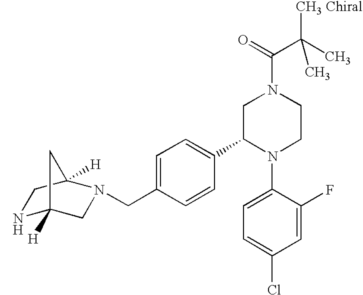 | 1.84* | 485^{a} | A to G then K to L |
| 33 | 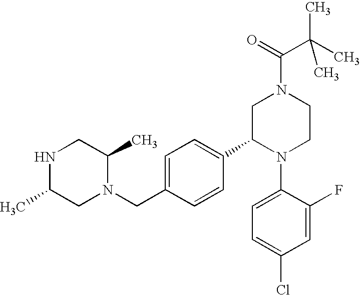 | 2.01* | 501.1^{a} | A to G then K to L |
| 34 | 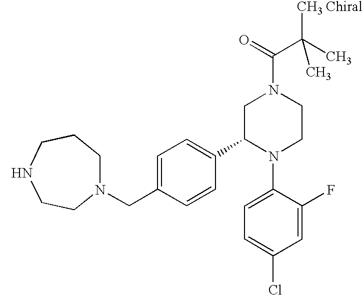 | 1.80* | 487.1^{a} | A to G then K to L |
| 35 | 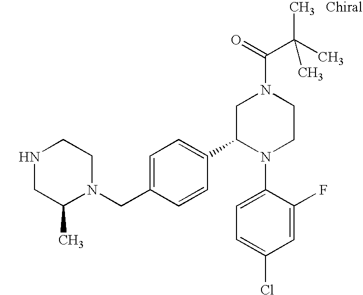 | 1.98* | 487.1^{a} | A to G then K to L |
| 36 | 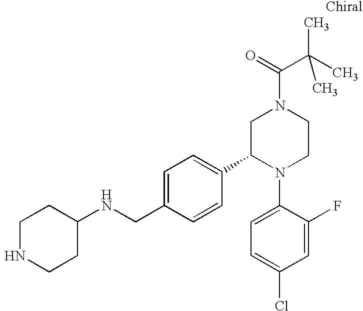 | 1.69* | 487.1^{a} | A to G then K to L |
| 37 | 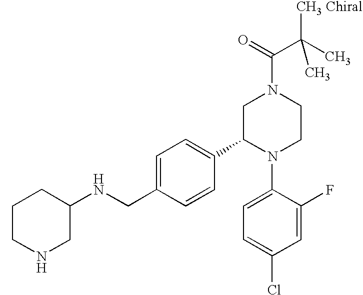 | 1.76* | 487.1^{a} | A to G then K to L |
| 38 | 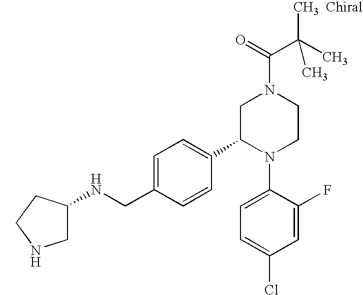 | 1.74* | 473^{a} | A to G then K to L |
| 39 | 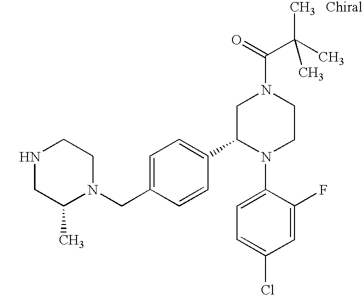 | 1.97* | 487.1^{a} | A to G then K to L |
| 40 | 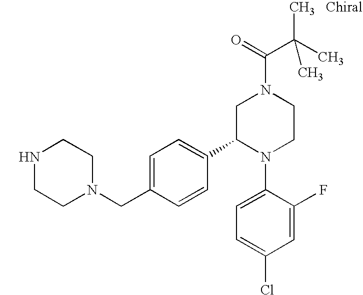 | 1.94* | 473^{a} | A to G then K to L |
| 41 | 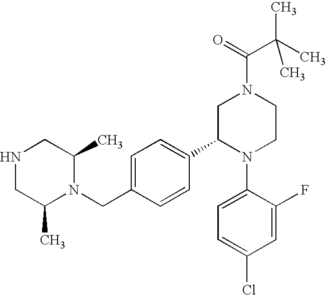 | 2.04* | 501.1^{a} | A to G then K to L |
| 42 | 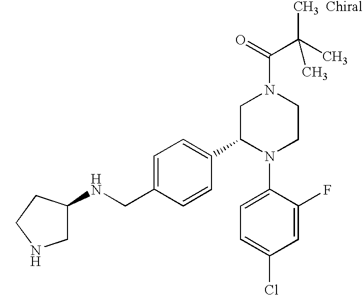 | 1.72* | 473^{a} | A to G then K to L |
| 43 | 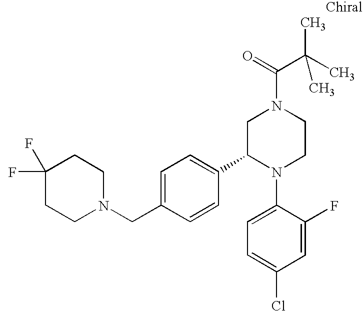 | 2.26* | 508^{a} | A to G then K to L |
| 44 | 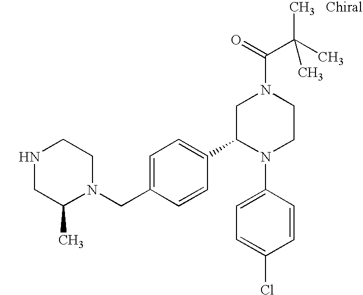 | 1.91* | 469.1^{a} | A to G then K to L |
| 45 | 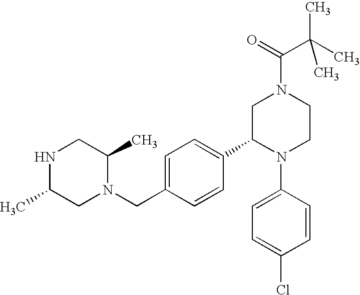 | 1.93* | 483.1^{a} | A to G then K to L |
| 46 | 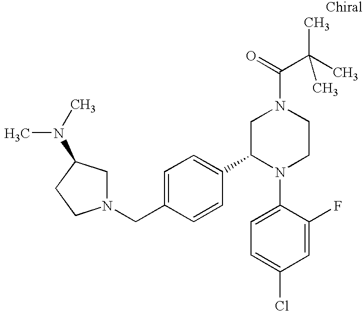 | 1.82* | 501.1^{a} | A to G then K to L |
| 47 | 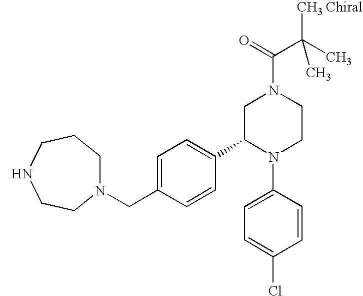 | 1.73* | 469.1^{a} | A to G then K to L |
| 48 | 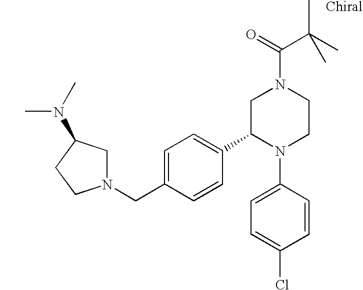 | 1.77* | 483.1^{a} | A to G then K to L |
| 49 | 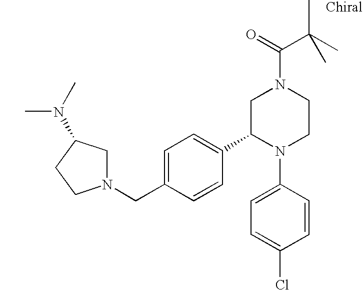 | 1.77* | 483.1^{a} | A to G then K to L |
| 50 | 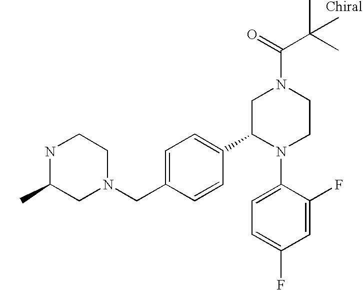 | 3.31* | 470.6 | A to G then K to L |
| 51 | 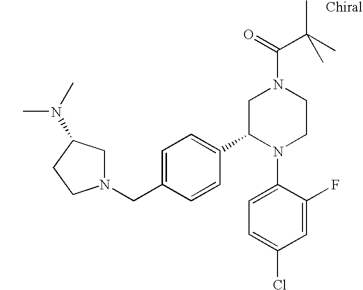 | 1.82* | 501.1^{a} | A to G then K to L |
| 52 | 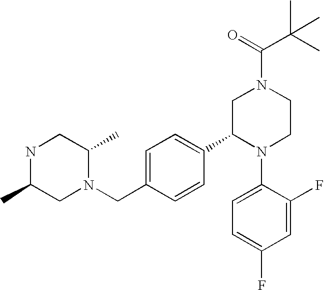 | 3.31^{@} | 484.6 | A to G then K to L |
| 53 | 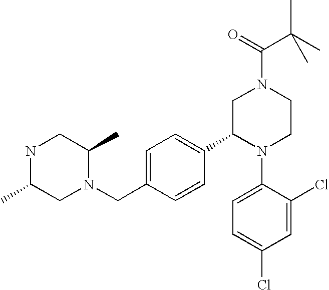 | 2.10* | 517.5^{b} | A to G then K to L |
| 54 | 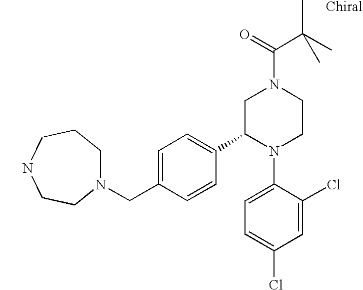 | 1.87* | 503.5^{b} | A to G then K to L |
| 55 | 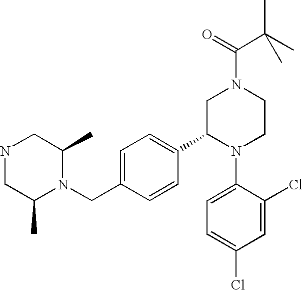 | 2.11* | 517.5^{b} | A to G then K to L |
| 56 | 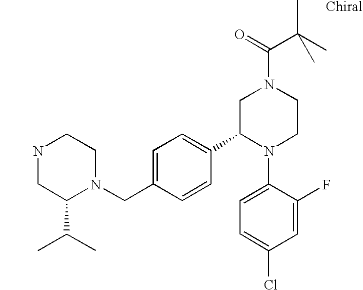 | 2.13* | 515.1^{a} | A to G then K to L |
| 57 | 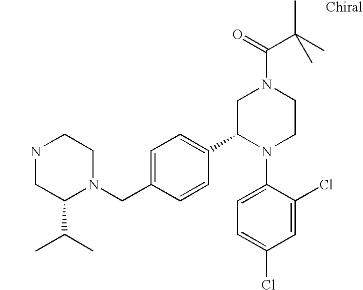 | 2.20* | 531.6^{b} | A to G then K to L |
| 58 | 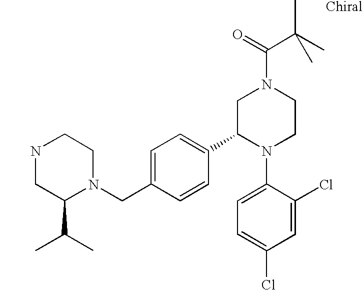 | 2.20* | 531.6^{b} | A to G then K to L |
| 59 | 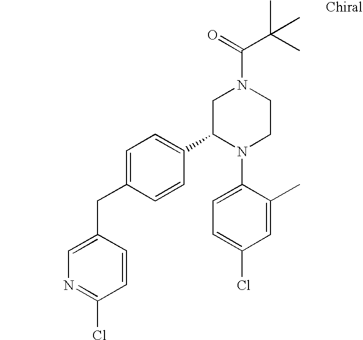 | 4.33^{@} | 496.5^{a} | A to G then M |
| 60 | 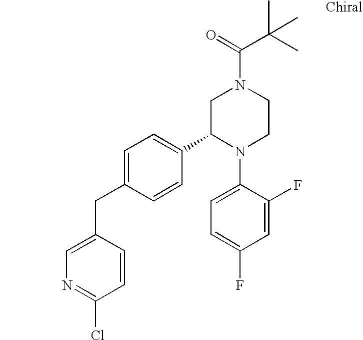 | 4.20^{@} | 484 | A to G then M |
| 61 | 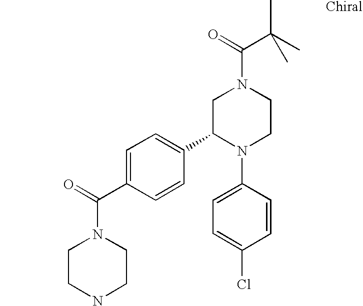 | 1.92^{@} | 469^{a} | A to G then N |
| 62 | 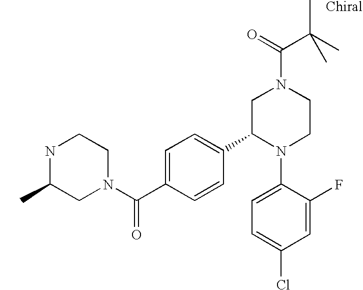 | 1.90* | 501^{a} | A to G then N |
| 63 | 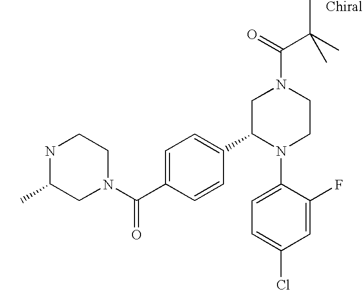 | 1.92* | 501^{a} | A to G then N |
| 64 | 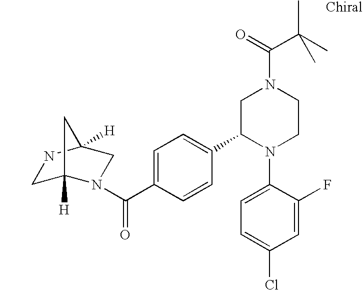 | 1.97* | 499^{a} | A to G then N |
| 65 | 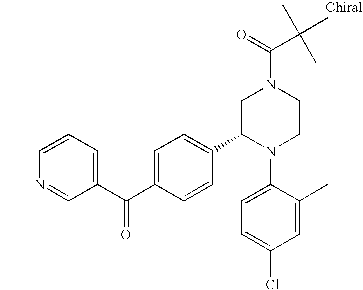 | 4.03^{@} | 476^{a} | A to G then O |
| 66 | 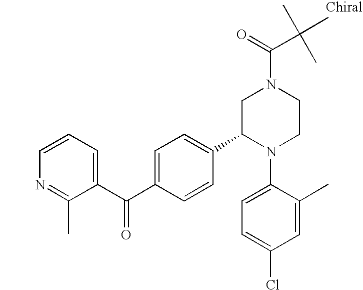 | 4.08^{@} | 490^{a} | A to G then O |
| 67 | 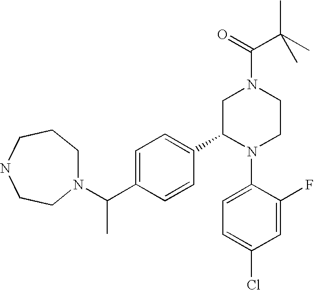 | 1.80* | 501.1^{a} | A to G then P |
| 68 | 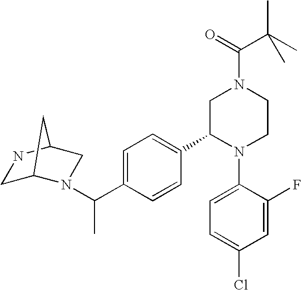 | 1.89* | 499.1^{a} | A to G then P |
| 69 | 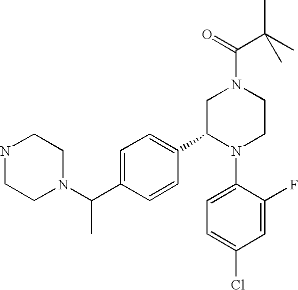 | 1.95* | 487.1^{a} | A to G then P |

^{a}Chlorine pattern detected
^{b}Dichlorine pattern detected
^{c}Bromine-chlorine pattern detected
*LCMS method A;
^{#}LC/MS method B;
^{@}LC/MS method C

In the examples, characterization and/or purification were performed using standard spectroscopic and chromatographic techniques, including liquid chromatography-mass spectroscopy (LC-MS) and high performance liquid chromatography (HPLC), using the conditions described in methods A to C. NMR experiments were conducted on a Bruker DPX400 ultra shield NMR spectrometer in the specified solvent. Reactions carried out under microwave irradiation were conducted in a Smith Synthesizer or a CEM Discovery Microwave.

### LCMS Method A

### Instrument: HP1100

Column: Luna 3 μm, C18(2), 30 mm×4.6 mm i.d. from Phenomenex

### Temperature: 22° C.

Solvents: A—Water+10 mmol/L ammonium acetate+0.08% (v/v) formic acid
B—95% Acetonitrile−5% Solvent A+0.08% (v/v) formic acid

### Gradient:

| | | | Flow |
|---|---|---|---|
| Time (min) | Solvent A (%) | Solvent B (%) | (cm³min⁻¹) |
| 0 | 95 | 5 | 2 |
| 0.25 | 95 | 5 | 2 |
| 2.50 | 5 | 95 | 2 |
| 2.55 | 5 | 95 | 3 |
| 3.60 | 5 | 95 | 3 |
| 3.65 | 5 | 95 | 2 |
| 3.70 | 5 | 95 | 2 |
| 3.75 | 95 | 5 | 2 |

Detection: UV detection at 230, 254 and 270 nm
Mass Spec: HP1100 MSD, series A
Ionization was positive or negative ion electrospray
Molecular weight scan range was 120-1000

### LCMS Method B

Instrument: Waters 2695 pump module and 2700 sample manager
Column: Gemini 5 μm, C18 110A, 30 mm×2 mm i.d. from Phenomenex. Pt no 00A-4435-B0
Temperature: 22° C.
Solvents: A—Water+10 mmol/ammonium formate+0.08% (v/v) formic acid at pH 3.5
   B—100% Acetonitrile+0.025% (v/v) formic acid
Injection Volume 5 uL
Gradient:

| | | | Flow |
|---|---|---|---|
| Time (min) | Solvent A (%) | Solvent B (%) | (cm³min⁻¹) |
| −1.0 (Equil) | 95 | 5 | 1.2 |
| 0 | 95 | 5 | 0.8 |
| 0.25 | 95 | 5 | 0.8 |
| 2.50 | 5 | 95 | 0.8 |
| 4.0 | 5 | 95 | 0.8 |
| 5 | 5 | 95 | 1.0 |
| 5.2 | 95 | 5 | 1.0 |

Detection: UV detection from 220 to 400 nm (1:3 split)
Mass Spec Waters ZQ2000, M/z range 100 to 900

### LCMS Method C

Instrument: Waters FractionLynx MS autopurification system
Column: Luna 5 μm, C18(2), 100 mm×21.2 mm i.d. from Phenomenex
Temp: ambient
Solvents: A—water+0.08% (v/v) formic acid
B—95% methanol-water+0.08% (v/v) formic acid
   Flow rate: 20 cm³ min⁻¹

### Gradient:

| Time (min) | Solvent A (%) | Solvent B (%) |
|---|---|---|
| 0 | 95 | 5 |
| 0.5 | 50 | 50 |
| 7.0 | 20 | 80 |
| 7.5 | 5 | 95 |
| 9.5 | 5 | 95 |
| 10.0 | 95 | 5 |

Detection: Photodiode array 210 to 400 nm
Mass spec: MicroMass ZQ
Ionization was positive or negative ion electrospray
Molecular weight scan range was 150-1000
Collection: Triggered on selected mass ion

### In Vitro Functional Test

Cannabinoid receptors are members of the super family of G protein-coupled receptors. [³⁵S]GTPγS is a non-hydrolysable GTP analogue and allows the exchange of GDP for radio labelled-GTP on the alpha subunit of the associated G-protein. Receptor activation or function, of a CB₁ membrane preparation, can therefore be followed quantitatively by determining the amount of radioactivity associated with the membranes. This can be used to determine the level of functional effect of any given ligand, yielding agonist, antagonist or inverse agonist data. The effect of compounds on CB1 receptor mediated accumulation of [³⁵S]GTPγS binding was assessed by a modification of the method of Griffin et al (1998). Briefly cell membranes from cells expressing human recombinant CB1 receptor were purchased from Perkin Elmer (Cat No RBHCB1). Membranes were suspended in HEPES buffer, containing NaCl (100 mM), MgCl₂, (32 mM) Assays were incubated for 60 minutes in a final volume of 250 □l, containing [³⁵S]GTPγS (1 nM, 1101 Ci/mmol), 1 □M GDP and test compound. Test compounds were dissolved in DMSO at 10⁻² M and diluted subsequently in buffer containing 1% DMSO. Compounds were tested over the molar concentration range 10⁻¹⁰ to 10⁻⁵.

Evaluation of cannabinoid receptor agonists and antagonists using the guanosine-5′-O-(3-[35S]thio)-triphosphate binding assay in rat cerebellar membranes. Griffin, Graeme; Atkinson, Peter J.; Showalter, Vincent M.; Martin, Billy R.; Abood, Mary E. Journal of Pharmacology and Experimental Therapeutics (1998), 285(2), 553-560.

All compounds exemplified have affinities for the human cannabinoid CB1 receptor of between 0.5 nanomolar and 1 micromolar. By way of example, the compound described as Example 1 has an affinity of 12 nanomolar at human CB1 receptors.

Affinity at human CB2 receptors is generally greater than 1 micromolar for all compounds. For example, the compound described as Example 1 has an affinity of 2.87 micromolar at human CB2 receptors.

1. A compound of formula (I) or a pharmaceutically acceptable salt, hydrate or solvate thereof 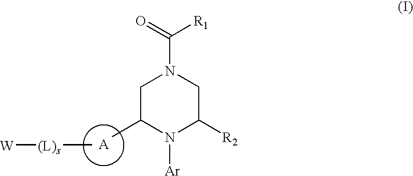
wherein
R₁ is a radical of formula -(Alk¹)ₘ-(NH)ₚ-(Alk²)ₙ-Q wherein
m, n and p are independently 0 or 1,
Alk¹ and Alk² are straight or branched chain divalent C₁-C₆ alkylene or C₂-C₆ alkenylene radicals, and
Q is (i) hydrogen, except in the case where m, n and p are each 0, or (ii) an optionally substituted carbocyclic or heterocyclic group;
R₂ is hydrogen, C₁-C₃ alkyl, cyclopropyl, or CF₃;
Ring A is a phenyl or 5- or 6-membered heteroaryl ring either of which is optionally substituted;
Ar is a phenyl or 5- or 6-membered heteroaryl ring either of which is optionally substituted;
L is —CH₂—, —C(═O)—, —NH—, —O—, —S—, —SO—, —SO₂—, —(CH₂)₂—, —CH═CH—, —OCH₂—, —CH(CH₃)—, or —NH—CH₂—;
s is 1 and W is an optionally substituted N-containing heterocyclic ring of 5 to 7 ring atoms; or s is 0 and W is an optionally substituted N-containing saturated heterocyclic ring of 5 to 7 ring atoms, or an N-containing heteroaryl ring of 5 or 6 ring atoms substituted by at least one substituent selected from amino, C₁-C₆ alkylamino or cyano.

2. A compound of formula (I) as set forth in claim 1 or a pharmaceutically acceptable salt, hydrate or solvate thereof, wherein R₁, R₂, ring A and Ar are as defined in claim 1;
L is —CH₂—, —C(═O)—, —NH—, —O—, —S—, —SO—, —SO₂—, —(CH₂)₂—, —CH═CH—, or —OCH₂—; and
s is 1 and W is an optionally substituted N-containing heterocyclic ring of 5 or 6 ring atoms; or s is 0 and W is an optionally substituted N-containing saturated heterocyclic ring of 5 or 6 ring atoms, or an N-containing heteroaryl ring of 5 or 6 ring atoms substituted by at least one substituent selected from amino, C₁-C₆ alkylamino or cyano.

3. A compound as claimed in claim 1 wherein p is 1.

4. A compound as claimed in claim 3 wherein m is 1 and -Alk¹- is —CH₂—,
—CH₂CH₂—, —CH(CH₃)CH₂—, or —C(CH₃)₂CH₂—.

5. A compound as claimed in claim 3 wherein m is 0.

6. A compound as claimed in claim 3 wherein -Alk²-Q is methyl, ethyl, n- or iso-propyl, or n-, sec-, or t-butyl.

7. A compound as claimed in claim 1 wherein p is 0.

8. A compound as claimed in claim 7 wherein Q is cyclopropyl, cyclopentyl, cyclobutyl, cyclohexyl, phenyl, N-piperidinyl, N-piperazinyl, N-morpholinyl, pyridyl, thienyl, furanyl or pyrrolyl.

9. A compound as claimed in claim 8 wherein m and n are both 0.

10. A compound as claimed in claim 7 wherein m is 0 and Q is hydrogen.

11. A compound as claimed in claim 1 wherein R₁ is —C(CH₃)₃ or
—NHC(CH₃)₃.

12. A compound as claimed in claim 1 wherein R₂ is hydrogen.

13. A compound as claimed in claim 1 wherein R₂ is methyl or trifluoromethyl.

14. A compound as claimed in claim 1 wherein Ar is phenyl or pyridyl which is substituted in the 4-position of the ring relative to the ring's point of attachment to the piperazine nitrogen.

15. A compound as claimed in claim 1 wherein optional substituents in Ar are selected from chloro, fluoro, bromo, methyl, trifluoromethyl, methoxy, trifluoromethoxy, cyano, amido, ester, phenyl, pyridyl, or pyrimidinyl.

16. A compound as claimed claim 1 wherein Ar is phenyl substituted in the 4 position by chloro, fluoro, bromo, methyl, trifluoromethyl, or cyano.

17. A compound as claimed in claim 1 wherein Ar is phenyl with a substituent in the 2-position selected from chloro, fluoro, bromo, methyl, trifluoromethyl, methoxy, trifluoromethoxy, cyano, amido, or ester.

18. A compound as claimed in claim 1 wherein ring A is selected from optionally substituted phenyl or pyridyl.

19. A compound as claimed in claim 18 wherein ring A is a phenyl ring, optionally substituted by one or two substituents selected from chloro, fluoro, bromo, methyl, trifluoromethyl, methoxy, trifluoromethoxy, or cyano.

20. A compound as claimed in claim 1 wherein ring A is 1,4-phenylene.

21. A compound as claimed in claim 1 wherein s is 1 and L is —CH₂—, —CH(CH₃)—, or —NH—CH₂—.

22. A compound as claimed in claim 1 wherein s is 0.

23. A compound as claimed in claim 21 wherein W is optionally substituted pyrrolidinyl, piperidinyl, piperazinyl, homopiperazinyl, morpholinyl, pyridinyl or pyrimidinyl.

24. A compound as claimed in claim 23 wherein W is pyrrolidin-3-yl, piperidin-3-yl, piperidin-4-yl, piperazin-1-yl or homopiperazin-1-yl.

25. A compound as claimed in claim 2 wherein s is 1 and L is —CH₂—, —C(═O)—, or —NH—.

26. A compound as claimed in claim 2 wherein s is 0.

27. A compound as claimed in claim 25 wherein W is optionally substituted pyrrolidinyl, piperidinyl, piperazinyl, morpholinyl, pyridinyl or pyrimidinyl.

28. A compound as claimed in claim 27 wherein W is 3-(dimethylamino)-pyrrolidin-1-yl, piperidin-4-yl, 4,4-difluoro-piperidin-1-yl, piperazin-1-yl, 1-methyl-piperazin-4-yl, 1-(tertiarybutyloxycarbonyl)-piperazin-4-yl, 1-(pyridin-4-yl)-piperazin-4-yl, 2-isopropyl-piperazin-1-yl, 2-methyl-piperazin-1-yl, 3-methyl-piperazin-1-yl, 2,6-dimethyl-piperazin-1-yl, 3,5-dimethyl-piperazin-1-yl, 2,5-dimethyl-piperazin-1-yl, morpholin-4-yl, pyridin-4-yl, pyridin-3-yl, 3-methyl-pyridin-4-yl, 2-methyl-pyridin-3-yl, or 2-chloro-pyridin-5-yl.

29. A compound as claimed in claim 26 wherein W is optionally substituted pyrrolidinyl, piperidinyl, piperazinyl or morpholinyl.

30. A compound as claimed in claim 29 wherein W is 3-(dimethylamino)-pyrrolidin-1-yl, piperidin-4-yl, 4,4-difluoro-piperidin-1-yl, piperazin-1-yl, 1-methyl-piperazin-4-yl, 1-(tertiarybutyloxycarbonyl)-piperazin-4-yl, 1-(pyridin-4-yl)-piperazin-4-yl, 2-isopropyl-piperazin-1-yl, 2-methyl-piperazin-1-yl, 3-methyl-piperazin-1-yl, 2,6-dimethyl-piperazin-1-yl, 3,5-dimethyl-piperazin-1-yl, 2,5-dimethyl-piperazin-1-yl, or morpholin-4-yl.

31. A compound as claimed in claim 26 wherein W is pyridyl or pyrimidinyl substituted by at least one substituent selected from amino, C₁-C₆ alkylamino or cyano.

32. A compound as claimed in claim 31 wherein the pyridyl or pyrimidinyl ring is ortho-substituted relative to its point of attachment.

33. A compound as claimed in claim 31 wherein W is optionally substituted by halo, C₁-C₆ alkyl, C₁-C₆ alkoxy, or halo C₁-C₆ alkyl.

34. A compound as claimed in claim 31 wherein W is 2-amino-pyridin-3-yl, 2-cyano-pyridin-3-yl, 3-amino-pyridin-4-yl, 3-cyano-pyridin-4-yl, 2-amino-4-chloro-pyrimidin-6-yl, 2-amino-4-methyl-pyrimidin-6-yl, or 4-amino-6-methyl-pyrimidin-5-yl.

35. A pharmaceutical composition comprising a compound as claimed in claim 1 and a pharmaceutically acceptable carrier.

36. (canceled)

37. A method of treatment of a mammal suffering from a condition responsive to inhibition of CB1 activity, comprising administering to the mammal an amount of a compound as claimed in claim 1 effective to inhibit CB1 activity in the mammal.

38. The method as claimed in claim 37 wherein the condition responsive to inhibition of CB1 activity is selected from psychosis, memory deficit, cognitive disorders, attention deficit disorder, migraine, neuropathy, neuro-inflammatory disorders, cerebral vascular injuries, head trauma, anxiety disorders, depression, stress, epilepsy, dementia, distonia, Alzheimer's disease, Huntingdon's disease, Tourette's syndrome, ischaemia, pain, Parkinson's disease, schizophrenia, substance abuse disorders, smoking cessation, treatment of nicotine dependence and/or treatment of symptoms of nicotine withdrawal, gastrointestinal disorders, eating disorders associated with excessive food intake, non-insulin dependant diabetes mellitus, bone resorption, osteoporosis, bone cancer or Paget's disease of bone.

39. A method as claimed in claim 38 for abuse of nicotine, alcohol and/or opiates.

40. A method as claimed in claim 38 for obesity.

41. A method as claimed in claim 38 for Parkinson's Disease.

42. A method as claimed in claim 38 for smoking cessation.

43. A method as claimed in claim 38 for gastrointestinal disorders.

44. A method as claimed in claim 38 for a cognitive disorder.

45. A method as claimed in claim 38 for non-insulin dependent diabetes mellitus.
